(19) United States
(12) Patent Application Publication
(10) Pub. No.: US 2013/0053392 A1
(43) Pub. Date: Feb. 28, 2013

(54) CARBONIC ANHYDRASE INHIBITORS

(71) Applicants: Peter Ebbesen, Hojbjerg (DK); Claudlu T. Supuran, Florence (IT); Andrea Scozzafava, Florence (IT); Erik Olai Pettersen, Oslo (NO); Kaye Williams, Manchester (GB); Ludwig Dubois, Bilzen-Rosmeer (BE); Philippe Lambin, Bousval (BE)

(72) Inventors: Peter Ebbesen, Hojbjerg (DK); Claudlu T. Supuran, Florence (IT); Andrea Scozzafava, Florence (IT); Erik Olai Pettersen, Oslo (NO); Kaye Williams, Manchester (GB); Ludwig Dubois, Bilzen-Rosmeer (BE); Philippe Lambin, Bousval (BE)

(21) Appl. No.: 13/578,654

(22) PCT Filed: Feb. 14, 2011
(86) PCT No.: PCT/EP2011/052156

§ 371 Date: Oct. 31, 2012

(30) Foreign Application Priority Data
Feb. 12, 2010 (GB) 1002412.3
Oct. 11, 2010 (GB) 1017098.3

(51) Int. Cl. A61K 31/255; C07D 307/79; A61K 31/343; C07D 319/18; A61K 31/357; C07D 211/58; A61K 31/4468; C07D 213/89; A61K 31/44; C07D 241/04; A61K 31/495; C07C 311/37; C07D 311/88; A61P 35/00; A61P 35/04; A61K 31/18; G01N 21/00; C07C 307/02

(52) U.S. Cl. 514/252.12; 558/48; 514/517; 549/462; 514/469; 549/362; 514/452; 546/224; 514/329; 546/306; 514/353; 544/400; 564/56; 514/595; 549/225; 564/49; 514/597; 435/7.8

(57) ABSTRACT

A carbonic anhydrase IX (CA IX) inhibitor which comprises a compound of general formula: R—NH—CX—NH—(CH₂)ₙ—Ar-Q-SO₂—NH₂ or a pharmaceutically-acceptable salt, derivative or prodrug thereof; wherein n=0, 1 or 2; Q is O or NH; X is O or S; and R comprises an organic substituent group.

### FIELD OF THE INVENTION

The present invention relates to carbonic anhydrase inhibitors, their use in medicine including cancer treatment, pharmaceutical compositions containing such inhibitors and inhibitors for use in diagnosis or imaging.

### BACKGROUND OF THE INVENTION

As some solid cancer tumours grow in cancer patients, hypoxic regions may be formed, particularly in the interior of the tumour. These hypoxic regions therefore tend not to be associated with a blood supply. Hypoxic cancer cells represent a danger to cancer patients because there is an increased tendency for hypoxic tumour micro environments to stimulate metastatic progression and because hypoxic tumour cells have increased resistance to treatment. Chemotherapeutic agents have problems reaching the cells from the blood supply and the hypoxia itself protects cells against radiotherapy because oxygen is necessary for the cytotoxic action of radiation-generated free radicals. Tumour hypoxia is therefore generally associated with poor prognosis for cancer patients.

Carbonic anhydrases (CAs) are widespread zinc metalloenzymes found in higher vertebrates including humans. 16 isozymes have been characterised to date, many of which are involved in critical physiological processes. They catalyse the following reaction: CO₂+H₂O═H⁺+HCO₃⁻. In humans, CAs are present in a large variety of tissues including the gastrointestinal tract, the reproductive tract, the nervous system, kidneys, lungs, skin and eyes. The different isozymes are localised in different parts of the cell with CA I and CA II, important isozymes in normal cells, being localised in the cytosol.

The gene expression profile of a hypoxic cancer cell is different from that of other cancer cells in a normally-oxygenated environment (“normoxic conditions”). Under hypoxic conditions, transcription factor HIFα is sufficiently stable to give rise to hypoxia-induced gene expression. One consequence of this is that the distribution of isoforms of carbonic anhydrase (CA) is altered in hypoxic tumour cells as compared with normoxic cells. As a result, CA isozymes IX and XII are found to be overexpressed in hypoxic tumour cells. These isozymes have therefore become known as potential targets for anti-tumour therapy and imaging.

Unlike many CAs, CA IX and CA XII are both extracellularly localised on hypoxic tumour cells. These enzymes play a role in carbon fixation which may aid the growth of the tumour cells and also in acidification of the cells' micro environment. They are therefore thought to provide a target for cancer therapy because they are relatively specific to the hypoxic tumour cells and appear to be important in the survival and proliferation of those cells.

Efforts have been made to find inhibitors which are specific for CA IX and/or CA XII. CA IX is known to be a particularly catalytically efficient isozyme, having a k_{cat}=5.5×10⁵ s⁻¹ whereas CA XII has catalytic activity of one order of magnitude lower. A consequence of this is that for inhibitors to be effective they must have relatively low inhibition constants (Ki of the order of nanomolar). Furthermore, for such inhibitors to be useful, they must also be relatively specific for CA IX/CA XII as compared to the CA isozymes which are usually found distributed intracellularly in normal cells such as CA II. Winum et al describe in “Anti-Cancer Agents in Medicinal Chemistry, 2009, 9, 693-702” a variety of different CA IX inhibitors. The most widely studied CA IX inhibitors are those in the sulphonamide series, typically having the formula R—Ar—SO₂NH₂. Therapeutic and diagnostic agents which are sulphonamides are described in WO2006/137092 and sulphonamide-based metal chelate complexes for imaging are described in WO2009/089383. Great variation is reported in the CA IX inhibition constants for the sulphonamides as well as variation in the selectivity of the inhibitors. Sulphamate and sulphamide inhibitors have also been proposed in Winum et al. The best CA IX inhibitor was 4-chlorophenyl sulphamate. However, the usefulness of such an inhibitor for practical purposes as a pharmaceutically-active compound is limited because it is relatively unstable in solution.

There is a need in this art for new inhibitors for CA IX and CA XII for use in pharmaceutical applications including cancer therapy, diagnosis and imaging.

### SUMMARY OF THE INVENTION

Accordingly, in a first aspect, the present invention provides a carbonic anhydrase IX (CA IX) inhibitor which comprises a compound of general formula:

R—NH—CX—NH—(CH₂)ₙ—Ar-Q-SO₂—NH₂

or a pharmaceutically-acceptable salt, derivative or prodrug thereof; wherein

n=0, 1 or 2;

Q is O or NH;

X is O or S; and

R comprises an organic substituent group.

It has surprisingly been found that carbonic anhydrase inhibitors according to the invention are potent inhibitors of CA IX and selective for CA IX over CA II. The inhibitors may also inhibit CA XII. Although the inhibitors are sulphamates (Q=O) or sulphamides (Q=NH), they have been found to be relatively stable, particularly in solution, thereby enabling them to be used on a practical level in pharmaceutical compositions.

Carbonic anhydrases are thought to have a catalytic mechanism which relies upon an active site which contains a coordinated zinc ion. Inhibitors of the type according to the present invention are thought to act by forming an adduct between the zinc ion and the terminal nitrogen. The rest of the inhibitor molecule is accommodated in a binding pocket of the carbonic anhydrase enzyme which widens out to some extent as distance increases from the zinc ion. As a result, the binding pocket can accommodate a relatively wide variation in the organic substituent group R. The organic substituent group, which does not include H, may be aliphatic or aromatic and may include one or more heteroatoms. It is preferred that R is bulky and so short linear hydrocarbon groups are not preferred. Cyclic groups are preferred although acyclic groups having from 1 to 18 carbon atoms, including linear and/or branched chain, may be used as the organic substituent group.

The organic substituent group preferably comprises a substituted or unsubstituted cyclic substituent, which may be carbocyclic or heterocyclic. In one arrangement, the cyclic substituent comprises an aromatic substituent. Typically, the aromatic substituent has the formula Ar′—(CH₂)ₚ— in which Ar′ is a substituted or unsubstituted aromatic ring or ring system having up to 3 fused rings; p may be 0, 1 or 2 thereby allowing up to two methylene groups to link the aromatic ring or ring system to the rest of the molecule. Many of the Ar′ groups which have been found to be effective have a single aromatic ring such as a phenyl ring, thiophenyl ring or pyridyl or other 5-/6-membered heterocycles. Larger ring systems include naphthyl, benzofuranyl and benzodioxinyl. Alternatively, the aromatic substituent may have the formula Ar′R′—CH— in which R′ is Me and Ar′ is the same or different and is a substituted or unsubstituted aromatic ring or ring system as above. As a further alternative, R′ and Ar′ are each Ph wherein each Ph is the same or different and is substituted or unsubstituted.

The aromatic ring of the aromatic substituent may be substituted or unsubstituted. Typically, up to three substituents may be borne by each ring or ring system and these substituents include F, Cl, Br, I, CN, MeO, Ph, PhO, PhCH₂, NO₂, Me₂N, Me, EtO₂C, Ac, EtO, iPr, MeS and EtOOC. Aromatic substituents which have been found to be particularly effective may be selected from 4-F—C₆H₄, 4-Cl—C₆H₄, 4-Br—C₆H₄, 4-I—C₆H₄, 2,4,I₂—CH₆H₃, 2,4,6,I₃—C₆H₂, 4-NC—C₆H₄, 4-MeO—C₆H₄, 4-Ph-C₆H₄, 4-PhO—C₆H₄, C₆F₅, 4-PhCH₂—C₆H₄, 4-PhCH₂CH₂C₆H₄, 4-O₂N—C₆H₄, 4-Me₂N—C₆H₄, 2,3,4-F₃C₆H₂, 3,5-Me₂C₆H₃, 4-EtO₂C—C₆H₄, 1-naphthyl, 2-Br-4,6-F₂C₆H₂, 2,4,6-Cl₃C₆H₂, Ph, 3,4-Cl₂C₆H₃, 3-Cl—C₆H₄, 2,4-F₂C₆H₃, 2-Me-4-MeO—C₆H₃, 2-Ph—C₆H₄, 2-PhO—C₆H₄, 3-PhO—C₆H₄, 4-Ac—C₆H₄, 3-Ac—C₆H₄, 4-PhCH₂O—C₆H₄, 2-MeO-5-Me-C₆H₃, 2-EtO—C₆H₄, 4-MeC₆H₄—CH₂, Ph₂CH, 4-iPr—C₆H₄, 2-iPr—C₆H₄, fluoren-9-yl, 3-MeS-C₆H₄, 2-naphthyl, 2-EtOOC—C₆H₄, 3-(2,3-dihydrobenzofuran-5-yl), 3-EtOOC—C₆H₄, 2-NC—C₆H₄, 1-naphthyl-CH₂CH₂, thiophen-2-yl-CH₂CH₂, 3-(2,3-dihydro-benzo[1,4]dioxin-6-yl), furan-2-yl, 1-naphthyl-Me-CH, 3-NO₂—C₆H₄, 2,4(MeO)₂—C₆H₄, 2-Me-4Cl—C₆H₃, Ph-CH₂—CH₂, 4-BuO—C₆H₄, Ph-CH₂, 2-Me-C₆H₄, 2-Cl—C₆H₄, 4-HCOO—C₆H₄, pyridin-2-yl-methyl/ethyl and pyridin-4-yl-methyl-ethyl.

According to a further arrangement, the cyclic substituent may comprise an alicyclic substituent. This alicylic substituent may be substituted or unsubstituted and may be saturated or unsaturated. The alicyclic substituent may have a single ring or may have a plurality of fused rings. Preferably, the alicyclic substituent is substituted or unsubstituted 1- or 2-adamantyl, N-Boc-piperidin-4-yl, substituted or unsubstituted cyclohexyl, or substituted or unsubstituted piperazine-methyl/ethyl.

In a further arrangement, the organic substituents may comprise a charged moiety such as substituted pyridinium, piperidinium, or piperazinium or tetralkylammonium. Where the organic substituent is charged, this confers on the inhibitor a further advantage. A charged inhibitor cannot readily cross the cell membrane and so is prevented from penetrating the intracellular space. Such inhibitors are less likely to be metabolised before they reach their target cells because they will not enter the intracellular space of other cells, such as those surrounding the target cells. Such inhibitors are also selective for extracellular CAs because they will not enter the cells and bind to the intracellular CAs. Since aerobic/normoxic cells do not express extracellular drug-binding CA's the inhibitor will be able to diffuse freely through the aerobic region close to blood vessels without being bound there, and reach into the hypoxic regions.

The Ar group of the carbonic anhydrase inhibitors of the present invention denotes an aromatic group which typically has a single ring or two fused rings. The Ar group may be carbocyclic or heterocyclic and may be substituted or unsubstituted. Typically, small substituents are preferred such as Me, Et, OH, MeO, F, Cl, Br, I and CN. Whether or not the Ar group is substituted, two ring positions are taken up with Q and (CH₂)ₙ (which is a direct bond to the rest of the molecule when n=0). These two ring positions may be at any point on the Ar ring except ortho to one another because of steric constraints. For example, where Ar is a single ring such as phenyl, Q and (CH₂)ₙ are positioned meta or para to one another (i.e. 1, 3 or 1,4). Where Q is para to (CH₂)ₙ, small substituent groups may be positioned on the ring as described above. Where Q is meta to (CH₂)ₙ the above substituents may also be positioned on the ring as described above; additionally, in the para position, a larger substituent may be incorporated instead, such as a C1 to C5 hydrocarbyl substituent. Preferably, Ar is a substituted or unsubstituted phenylene or naphthalene, more preferably a phenylene group, most preferably praraphenylene.

In the inhibitors of the invention, the group (CH₂)ₙ acts as a linker between the NH—CX—NH group and the Ar group. Whilst there is some tolerance in the distance between NH—CX—NH and Ar, this is limited and so n is no greater than 2. It is preferred that n=0, thereby denoting a direct bond between NH—CX—NH and Ar. Where Ar is praraphenylene, this gives rise to the following structure:

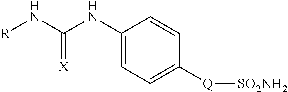

X may be O or S thus denoting a ureido or thioureido group.

Preferred sulfamate inhibitors according to the invention have the following general formula:

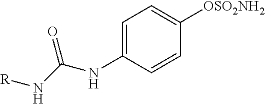

wherein R denotes the aromatic substituent and is selected from 4-F—C₆H₄, 4-Cl—C₆H₄, 4-Br—C₆H₄, 4-I—C₆H₄, 4-NC—C₆H₄, 4-MeO-C₆H₄, 4-Ph-C₆H₄, 4-PhO—C₆H₄, C₆F₅, 4-PhCH₂—C₆H₄, 4-PhCH₂CH₂C₆H₄, 4-O₂N—C₆H₄, 4-Me₂N—C₆H₄, 2,3,4-F₃C₆H₂, 3,5-Me₂C₆H₃, 4-EtO₂C—C₆H₄, 1-naphthyl, 2-Br-4,6-F₂C₆H₂, 2,4,6-Cl₃C₆H₂, Ph, 3,4-Cl₂C₆H₃, 3-Cl—C₆H₄, 2,4-F₂C₆H₃, 2-Me-4-MeO—C₆H₃, 2-Ph-C₆H₄, 2-PhO—C₆H₄, 3-PhO—C₆H₄, 4-Ac—C₆H₄, 3-Ac—C₆H₄, 4-PhCH₂O—C₆H₄, 2-MeO-5-Me-C₆H₃, 2-EtO—C₆H₄, 4-MeC₆H₄—CH₂, Ph₂CH, 4-iPr—C₆H₄, 2-iPr—C₆H₄, fluoren-9-yl, 3-MeS-C₆H₄, 2-naphthyl, 2-EtOOC—C₆H₄, 3-(2,3-dihydrobenzofuran-5-yl), 3-EtOOC—C₆H₄, 2-NC—C₆H₄, 1-naphthyl-CH₂CH₂, thiophen-2-yl-CH₂CH₂, 3-(2,3-dihydro-benzo[1,4]dioxin-6-yl), 1-adamantyl and N-Boc-piperidin-4-yl, pyridin-2-yl-methyl, pyridin-2-yl-ethyl; pyridin-4-yl-methyl-ethyl-, 4-N-methyl-piperazine-methyl/ethyl and the positively charged moieties shown below (A-C):

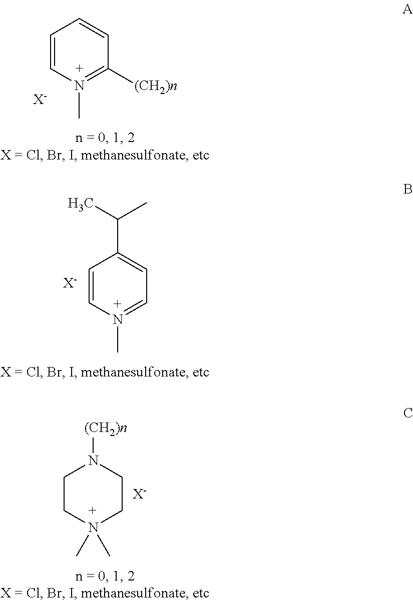

Other preferred sulfamate inhibitors have the formulae 15 to 18 or 20, as set out in Table 3 below.

Preferred sulfamides have the formulae 7a to 7p or 8a to 8h as set out in Table 2 below.

Whilst various CA IX inhibitors of the prior art have Ki values of the order of micromolar, inhibitors of the present invention have a Ki for CA IX of up to about 150 nM, usually up to about 120 nM and a corresponding Ki for CA XII of up to about 240 nM, usually up to about 80 nM. It is preferred that the Ki for CA IX is no greater than 50 nM, more preferably no greater than 30 nM, preferably no greater than 20 nM, more preferably no greater than 10 nM. The selectivity ratio of the inhibitors of the present invention as measured by Ki CA II/Ki CA IX can be at least 6 and is typically at least 7.5, advantageously at least 10, more advantageously at least 11.5, preferably at least 12.5, more preferably at least 15, still more preferably at least 20, yet still more preferably at least 30, most preferably at least 40 and especially at least 50. As it will be appreciated, the higher the value for the selectivity ratio, the less likely side effects may arise in the use of the inhibitors by virtue of their inhibition of cytosolic CA isozymes in normal cells.

Typically, CA inhibition is measured by assaying for CA-catalysed CO₂ hydration activity using an appropriate indicator dye. As described in further detail in the specific examples, phenol red may be used as the indicator and this has an absorbent maximum of 557 nm. Stopped flow spectrophotometry may used to measure the rate of hydration activity.

In a further aspect, pharmaceutical compositions may be formulated comprising a carbonic anhydrase inhibitor as described herein or a pharmaceutically-acceptable salt, ester, or prodrug thereof optionally incorporating a pharmaceutically-acceptable diluents, excipient or carrier (including combinations thereof). Pharmaceutically-acceptable salts are known in this technical field and include salts with acids or bases which are accepted for the formation of salts for pharmaceutical use. For example, where the carbonic anhydrase inhibitor bears a carboxylic acid group, such pharmaceutically-acceptable salts include those of non-toxic cations such as quaternary ammonium ions, alkali metals such as sodium or potassium and alkaline earth metals such as calcium. Organic bases may also be used, such as ethanolamine, pyridine, trimethylamine or triethylamine. Alternatively, acid addition salts may be formed by the use of pharmaceutically-acceptable non-toxic acids such as hydrochloric acid, nitric acid, sulphuric acid, phosphoric acid, oxalic acid, fumaric acid, maleic acid, succinic acid, acetic acid, citric acid, tartaric acid, carbonic acid or an amino acid. Other materials may be added to the pharmaceutical compositions depending on the intended route of administration to the subject. Such additional materials include solubilising agents, coating agents, lubricants, binders and suspending agents. Non-toxic carriers, diluents and excipients are described in standard textbooks such as Remington's Pharmaceutical Sciences, Mack Publishing Company.

Pharmaceutical compositions may contain a prodrug form of the carbonic anhydrase inhibitor which is intended to become active only when metabolised by the subject. Such prodrug forms include esters which can be hydrolyzed in vivo with the formation of the sulfamate/sulfamide inhibitors presented above.

The present invention is not limited in relation to the particular route of administration to the subject. This may depend in part upon which part of the body of the subject needs to be targeted as well as the tolerance of the carbonic anhydrase inhibitor molecule to that particular route of administration. Standard routes of administration include oral, buccal, sublingual, inhalation, topical (including ophthalmic), rectal, vaginal, nasal and parenteral (including intravenous, intraarterial, intramuscular, subcutaneous and intraarticular).

The precise form of pharmaceutical composition and dosage thereof will also be dependent upon the subject to be treated including body weight, route of administration and precise disease conditions.

Pharmaceutically-acceptable derivatives include esters, amides, salts and nanoparticles based on the sulfamates/sulfamides described herein.

As will be appreciated, inhibitors according to the present invention may be used in medicine, and have particular use in cancer treatment. Whilst treatment of hypoxic cancer tumours is important in itself, a subject with cancer is likely to need additional treatment such as chemotherapy or radiation therapy. Treatments of the hypoxic tumour alone may account for approximately 40% reduction in tumour volume. The remaining tumour volume is therefore preferably treated additionally with chemotherapy or radiation therapy appropriate to normoxic cells. Accordingly, in one aspect, the inhibitors according to the invention are provided for use in cancer treatment of a subject who is treated additionally with chemotherapy or radiation therapy. Such inhibitors include compound 3p.

A further major problem related to cancer therapy is the formation of distant metastases. These cannot be treated radically with radiotherapy or surgery and therefore systemic chemical treatment is needed. However, chemotherapeutic drugs usually have limited specificity for cancer cells and thus, their use is limited by severe side-effects. Although chemotherapy has a high curative rate for some small groups of patients and some palliative effect for several groups it is curative in less than 5 percent of cancer patients over-all. The metastases may be detectable at the time of the first diagnosis, but may also appear following successful treatment of the primary tumor with radiotherapy or surgery. Recent data indicate that hypoxia in the primary tumor is a driving force for formation of metastasis (Rofstad E. K.: Microenvironment-induced cancer metastasis. Int. J radiat. Biol. 76; (2000) 589-605). This is well explained with respect to post-treatment effects related to radiotherapy since hypoxic cells are resistant to radiation and therefore may survive that treatment. It has, however been shown that hypoxia can be a negative prognostic factor related to malignant progression even after primary tumor surgery (Hockel M., Schlesinger K., Aral B., Mitze M., Schaffer U., Vaupel P.: Association between tumor hypoxia and malignant progression in advanced cancer of the uterine cervix. Cancer Res. 56; (1996) 4509-4515). Thus, there is a need for a treatment modality which reduces metastasis by specifically killing the hypoxic sub-fraction of cancer cells. A specific effect on the hypoxic sub-population is expected to be cancer specific since tissue hypoxia is specific to solid cancers. Such treatment would be valuable even if it does not have a strong effect on the primary tumor since the localized cancer can often be removed by combination with radical treatments like surgery or radiotherapy. Accordingly a CAIX inhibitor may be used as an anti-metastatic. Preferred CAIX inhibitors are those described herein, such as compound 3p.

In a further aspect, a product is provided comprising a CA IX inhibitor according to the invention and a chemotherapeutic agent as a combined preparation for simultaneous, separate or sequential use in cancer treatment. In this way, a kit may be provided containing the present inhibitors and further chemotherapeutic agents typically in separate containers. Alternatively, where appropriate, the chemotherapeutic agent and inhibitor may be administered to the subject together. Preferred CA IX inhibitors are those described herein, such as compound 3p.

In a further aspect, the CA IX inhibitors as described herein may be used in the preparation of a medicament for treatment of cancer.

The CA IX inhibitors of the present invention may also be used in methods of diagnosis or imaging. For these applications, the inhibitor typically includes a label appropriate to the particular diagnosis or imaging method. Such labels include fluorescent labels, spin labels, radiolabels or heavy atoms.

The organic substituent R may therefore be tailored to accommodate such labels. If the organic substituent group is itself fluorescent, this may confer upon the inhibitor a fluorescent label suitable for the above methods. An example of such an inhibitor is compound 20 in which the organic substituent comprises a 3-hydroxy-6-oxo-6H-xanthen-9-yl group. In the case of a radiolabel, this may be incorporated in any one of the inhibitors of the present invention. Suitable radioactive isotopes for inclusion in the molecules include the standard nuclides, such as ¹⁸F, ¹¹C, ⁶⁴Cu, ^{99m}Tc, etc. as well as the non-standard ones, such as ⁴⁵Ti, ⁶⁰Cu, ⁶¹Cu, ⁶⁶Ga, ⁷²As, ⁷⁴As, ⁷⁶Br, ⁸⁶Y, ⁸⁹Zr, ^{94m}Tc and ¹²⁴I

Where a heavy atom such as Zn(II); Cu(II), Co(II); Al(III); Fe(II); Fe(III); Re(VII); Os(VIII), Ru(VIII) is to be incorporated, this will typically be done by using an organic substituent group which comprises a chelator such as EDTA, DTPA, IDA, cryptates, crown ethers, porphyrins, etc (as for example those describe in A. Scozzafava, L. Menabuoni, F. Mincione, C. T. Supuran, Carbonic anhydrase inhibitors. A general approach for the preparation of water soluble sulfonamides incorporating polyamino-polycarboxylate tails and of their metal complexes possessing long lasting, topical intraocular pressure lowering properties. J. Med. Chem. 2002, 45, 1466-1476.)

In a further aspect of the present invention, there is provided an imaging composition comprising such CA IX inhibitors and a suitable diluents, excipient or carrier. Such compositions are typically manufactured for injection or per os administration into the subject.

### BRIEF DESCRIPTION OF THE DRAWINGS

The present invention will now be described in further detail, by way of example only, with reference to the following examples and accompanying drawings in which:

FIG. 1 shows the structures of sulfamide inhibitors of the present invention;

FIG. 2 shows structures of further sulfamide inhibitors of the present invention;

FIG. 3 shows a graph of tumour volume against time for treatment using a CA IX inhibitor of the present invention;

FIG. 4 shows a graph of tumour volume against time for the treatment comparing CAIX inhibitor 3k with an inert vehicle;

FIG. 5 shows the number of metastatic MDA-231 clonogens within lung tissue comparing CAIX inhibitor 3k with vehicle;

FIG. 6 compares MDA-231 cell migration in inhibitor-treated cells to control cells;

FIG. 7 shows a graph of gap closure against CA IX inhibitors comparing hypoxic and normoxic activity;

FIG. 8 shows gap closure dose response of CA IX inhibitors according to the invention;

FIG. 9 shows effective CA IX inhibitor on spheroid cell growth in presence or absence of doxorubicin or radiation treatment;

FIG. 10 shows CA IX mRNA expression in EV/2 and 94/1 cells under normoxic and hypoxic conditions;

FIG. 11 shows CA IX protein expression levels in EV/2 and 94/1 cells under normoxic and hypoxic conditions;

FIG. 12 shows quantitative fluorescence analysis of a fluorescent CA IX inhibitor binding to EV/2 and 94/1 cells under normoxic, hypoxic and reoxygenated conditions;

FIG. 13 shows quantitative FACS analysis of a fluorescent CA IX inhibitor binding to EV/2 and 94/1 cells under normoxic, hypoxic and reoxygenated conditions;

FIG. 14 shows immunofluorescence analysis of a fluorescent CA IX inhibitor binding to EV/2 and 94/1 cells under normoxic, hypoxic and reoxygenated conditions;

FIG. 15 shows pixel quantification of immunofluorescence staining of fluorescent CA IX inhibitor binding to EV/2 and 94/1 cells under normoxic, hypoxic and reoxygenated conditions;

FIG. 16 shows the effects of low oxygen conditions on survival of EV/2 and 94/1 cells;

FIG. 17 shows the effect of different doses of irradiation on survival of EV/2 and 94/1 cells under normoxic and anoxic conditions; and

FIG. 18 shows the effect of a CA IX inhibitor according to the invention on the sensitivity of EV/2 and 94/1 cells to irradiation under normoxic and anoxic conditions.

### DETAILED DESCRIPTION OF THE INVENTION

### Examples

### Example 1

### Preparation of Ureido-Sulfamates with Strong CA IX/XII Inhibitory Activity and Antitumor Properties

### Chemistry.

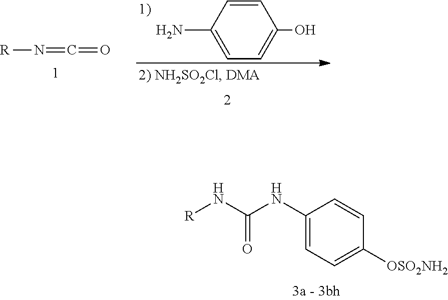

### Experimental Section:

### General.

All reagents and solvents were of commercial quality and used without further purification. All reactions were carried out under an inert atmosphere of nitrogen. TLC analyses were performed on silica gel 60 F₂₅₄ plates (Merck Art.1.05554). Spots were visualized under 254 nm UV illumination, or by ninhydrin solution spraying. Melting point were determined on a Büchi Melting Point 510 and are uncorrected. ¹H and ¹³C NMR spectra were recorded on Bruker DRX-400 spectrometer using DMSO-d₆ as solvent and tetramethylsilane as internal standard. Electron Ionization mass spectra (30 eV) were recorded in positive or negative mode on a Water MicroMass ZQ.

### Preparation of Sulfamates.

### General Procedure.

There are two procedures to achieve the first step depending on the substrate solubility.

### Procedure A (Non Soluble)

p-aminophenol 2 (1 equiv.) was added to a solution of isocyanate 1 (1 equiv.) in 15-20 ml of acetonitrile. The mixture was stirred at room temperature until complete formation of the product (TLC monitoring). The resulting precipitate was then filtered and washed with ethyl acetate several times.

### Procedure B (Soluble)

p-aminophenol 2 (1 equiv.) was added to a solution of isocyanate (1 equiv.) in 15-20 ml of acetonitrile. The mixture was stirred at room temperature until complete formation of the product (TLC monitoring). The mixture was then diluted with 100 ml of ethyl acetate and washed several times with water. When presence of p-aminophenol was detected by TLC, the organic phase was washed with an aqueous solution of HCl 1N, followed with brine. Finally the organic phase was dried over anhydrous magnesium sulfate and concentrated under vacuum.

The different ureas were controlled by ESI mass spectrometry and used in the next reaction without further purifications.

Sulfamates were then prepared by reacting the requisite phenol (1 equiv.) with sulfamoyl chloride (3 equiv.) in N,N-dimethylacetamide (Okada, M.; Iwashita, S. and Koizumi, N. Efficient general method for sulfamoylation of a hydroxyl group. Tetrahedron Lett. 2000, 41, 7057-7051.). (Sulfamoyl chloride was prepared from chlorosulfonyl isocyanate and formic acid as described previously: Appel, R. and Berger, G. Hydrazinsulfonsaüre-amide, I. Über das hydrazodisulfamid. Chem. Ber. 1958, 91, 1339-1341.). After completion of the reaction (TLC monitoring), the mixture was diluted with ethyl acetate and washed several times with water.

The organic extract was dried (MgSO₄) and concentrated under vacuum. The residue can be purified either by crystallization from ether/pentane or by chromatography on silica gel. Further details on the synthesis of membrane-impermeant inhibitors may be found in Scozzafava et al., J. Med. Chem., 2000, 43(2), 292-300.

The identity of each of the following compounds was confirmed by melting point analysis, ′H and ¹³C NMR, mass spectroscopy and elemental analysis (results not shown).
4-[(4-fluorophenyl)ureido]phenyl sulfamate 3a
4-[(4-chlorophenyl)ureido]phenyl sulfamate 3b
4-[(4-bromophenyl)ureido]phenyl sulfamate 3c
4-[(4-iodophenyl)ureido]phenyl sulfamate 3d
4-[(4-cyanophenyl)ureido]phenyl sulfamate 3e
4-[(4-methoxyphenyl)ureido]phenyl sulfamate 3f
4-[biphenyl-4-ylureido]phenyl sulfamate 3g
4-[(4-phenoxyphenyl)ureido]phenyl sulfamate 3h
4-[(pentafluorophenyl)ureido]phenyl sulfamate 3i
4-[benzylureido]phenyl sulfamate 3j
4-[phenethylureido]phenyl sulfamate 3k
4-[(4-nitrophenyl)ureido]phenyl sulfamate 3m
4-[(4-N,N-dimethylaminophenyl)ureido]phenyl sulfamate 3n
4-[(2,3,4-trifluorophenyl)ureido]phenyl sulfamate 3o
4-[(3,5-dimethylphenyl)ureido]phenyl sulfamate 3p
4-[(4-carboxyethylphenyl)ureido]phenyl sulfamate 3q
4-[(1-naphtyl)ureido]phenyl sulfamate 3r
4-[(2-bromo-4,6-difluorophenyl)ureido]phenyl sulfamate 3s
4-[(2,4,6-trichlorophenyl)ureido]phenyl sulfamate 3t
4-[(1-adamantyl)ureido]phenyl sulfamate 3u
4-[phenylureido]phenyl sulfamate 3v
4-[(3,4-dichlorophenyl)ureido]phenyl sulfamate 3x
4-[(3-chlorophenyl)ureido]phenyl sulfamate 3y
4-[(2,4-difluorophenyl)ureido]phenyl sulfamate 3z
4-[(4-methoxy-2-methylphenyl)ureido]phenyl sulfamate 3aa
4-[(biphenyl-2-yl)ureido]phenyl sulfamate 3ab
4-[(2-phenoxyphenyl)ureido]phenyl sulfamate 3ac
4-[(3-phenoxyphenyl)ureido]phenyl sulfamate 3ad
4-[(4-acetylphenyl)ureido]phenyl sulfamate 3ae
4-[(3-acetylphenyl)ureido]phenyl sulfamate 3af
4-[(4-benzyloxyphenyl)ureido]phenyl sulfamate 3ag
4-[(2-methoxy-5-methylphenyl)ureido]phenyl sulfamate 3ah
4-[(2-ethoxyphenyl)ureido]phenyl sulfamate 3ai
4-[(4-methylbenzyl)ureido]phenyl sulfamate 3aj
4-[(3-benzhydryl)ureido]phenyl sulfamate 3ak
4-[(4-isopropylphenyl)ureido]phenyl sulfamate 3am
4-[(2-isopropylphenyl)ureido]phenyl sulfamate 3an
4-[3-(9H-Fluoren-9-yl)ureido]phenyl sulfamate 3ao
4-[(3-thiomethoxy)ureido]phenyl sulfamate 3ap
4-[(2-naphtyl)ureido]phenyl sulfamate 3aq
4-[(2-carboxyethylphenyl)ureido]phenyl sulfamate 3ar
4-[3-(2,3-dihydrobenzofuran-5-yl)ureido]phenyl sulfamate 3 as
4-[(3-carboxyethylphenyl)ureido]phenyl sulfamate 3 at
4-[(2-cyanophenyl)ureido]phenyl sulfamate 3au
4-[(3-methoxyphenyl)ureido]phenyl sulfamate 3av
4-[3-(1-naphthalen-1-yl-ethyl)ureido]phenyl sulfamate 3ax
4-[3-(2-thiophen-2-yl-ethyl)ureido]phenyl sulfamate 3ay
4-[3-(2,3-dihydro-benzo[1,4]dioxin-6-yl)ureido]phenyl sulfamate 3az
4-[4-((N-benzyloxycarbonyl)piperidinyl)ureidomethyl]phenyl sulfamate 3aw
4-[pyridin-2-yl-methyl)ureido]phenyl sulfamate 3ba
4-[pyridin-2-yl-ethyl)ureido]phenyl sulfamate 3bb
4-[(N-methylpyridinium-2-yl-methyl)ureido]phenyl sulfamate iodide 3bc
4-[(N-methylpyridinium-2-yl-ethyl)ureido]phenyl sulfamate iodide 3bd
4-[pyridin-4-yl-methyl-ethyl)ureido]phenyl sulfamate 3be
4-[4-(N-methyl-pyridinium-4-yl-methyl-ethyl)ureido]phenyl sulfamate iodide 3bf
4-[(4-N-methyl-piperazine-ethyl)ureido]phenyl sulfamate 3bg
4-[(4,4-N-dimethyl-piperazinium-ethyl)ureido]phenyl sulfamate iodide 3bh

### Example 2

### Inhibition Studies on Carbonic Anhydrases

Inhibition studies were performed on carbonic anhydrases using the compounds prepared in accordance with Example 1. The inhibition constant (Ki) was determined for CA I, CA II, CA IX and CA XII using each of the prepared compounds. This is set out in further detail below and the results are presented in Table 1.

TABLE 1
CA inhibition data with the compounds described in the patent
3a-3bh

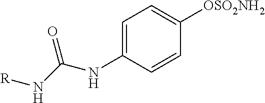

| | | | Ki (nM) | | | KiCAII/ |
|---|---|---|---|---|---|---|
| No | R | hCA I | hCA II | hCAIX | hCA XII | KiCAIX |
| 3a | 4-F—C₆H₄ | 2800 | 287 | 13 | 9 | 22.1 |
| 3b | 4-Cl—C₆H₄ | 2870 | 291 | 12 | 5 | 24.3 |
| 3c | 4-Br—C₆H₄ | 3050 | 305 | 13 | 8 | 23.5 |
| 3d | 4-I—C₆H₄ | 2100 | 186 | 10 | 10 | 18.6 |
| 3e | 4-NC—C₆H₄ | 3280 | 279 | 9 | 6 | 31.0 |
| 3f | 4-MeO—C₆H₄ | 2350 | 413 | 15 | 3 | 27.5 |
| 3g | 4-Ph—C₆H₄ | 5400 | 284 | 24 | 12 | 11.8 |
| 3h | 4-PhO—C₆H₄ | 4360 | 319 | 27 | 8 | 11.8 |
| 3i | C₆F₅ | 3180 | 145 | 6 | 1 | 24.2 |
| 3j | 4-PhCH₂—C₆H₄ | 6500 | 286 | 16 | 5 | 17.9 |
| 3k | 4-PhCH₂CH₂ | 5460 | 213 | 18 | 7 | 11.8 |
| 3m | 4-O₂N—C₆H₄ | 1230 | 450 | 6 | 4 | 75 |
| 3n | 4-Me₂N—C₆H₄ | 4370 | 348 | 9 | 2 | 42.7 |
| 3o | 2,3,4-F₃C₆H₂ | 3500 | 286 | 5 | 3 | 57.2 |
| 3p | 3,5-Me₂C₆H₃ | 5600 | 546 | 7 | 2 | 78 |
| 3q | 4-EtO₂C—C₆H₄ | 2450 | 431 | 8 | 6 | 53.9 |
| 3r | 1-naphthyl | 8700 | 298 | 17 | 18 | 17.5 |
| 3s | 2-Br-4,6-F₂C₆H₂ | 1390 | 641 | 9 | 6 | 71.2 |
| 3t | 2,4,6-Cl₃C₆H₂ | 3240 | 338 | 11 | 5 | 30.7 |
| 3u | 1-adamantyl | 43000 | 467 | 21 | 46 | 22.2 |
| 3v | Ph | 3240 | 393 | 16 | 10 | 24.6 |
| 3x | 3,4-Cl₂C₆H₃ | 3300 | 285 | 10 | 4 | 28.5 |
| 3y | 3-Cl—C₆H₄ | 4320 | 280 | 8 | 5 | 35 |
| 3z | 2,4-F₂C₆H₃ | 2450 | 192 | 7 | 2 | 27.4 |
| 3aa | 2-Me-4-MeO—C₆H₃ | 2960 | 119 | 10 | 6 | 11.9 |
| 3ab | 2-Ph—C₆H₄ | 8600 | 761 | 59 | 46 | 12.9 |
| 3ac | 2-PhO—C₆H₄ | 9000 | 815 | 78 | 69 | 10.5 |
| 3ad | 3-PhO—C₆H₄ | 10540 | 902 | 115 | 78 | 7.8 |
| 3ae | 4-Ac—C₆H₄ | 4520 | 348 | 13 | 4 | 26.8 |
| 3af | 3-Ac—C₆H₄ | 3480 | 413 | 18 | 9 | 22.9 |
| 3ag | 4-PhCH₂O—C₆H₄ | 6640 | 285 | 28 | 13 | 10.2 |
| 3ah | 2-MeO-5-Me—C₆H₃ | 3320 | 347 | 36 | 6 | 9.6 |
| 3ai | 2-EtO—C₆H₄ | 4500 | 486 | 15 | 4 | 32.4 |
| 3aj | 4-MeC₆H₄—CH₂ | 3600 | 310 | 10 | 10 | 31 |
| 3ak | Ph₂CH | 8690 | 459 | 54 | 59 | 8.5 |
| 3am | 4-iPr—C₆H₄ | 7510 | 613 | 30 | 18 | 20.4 |
| 3an | 2-iPr—C₆H₄ | 6540 | 547 | 67 | 37 | 8.2 |
| 3ao | fluoren-9-yl | 13000 | 750 | 75 | 26 | 10 |
| 3ap | 3-MeS—C₆H₄ | 3480 | 344 | 9 | 4 | 38.2 |
| 3aq | 2-naphthyl | 12500 | 568 | 16 | 29 | 35.5 |
| 3ar | 2-EtOOC—C₆H₄ | 3100 | 435 | 8 | 8 | 54.4 |
| 3as | 3-(2,3-Dihydro- | 2490 | 306 | 7 | 3 | 43.7 |
| | benzofuran-5-yl) | | | | | |
| 3at | 3-EtOOC—C₆H₄ | 4530 | 448 | 12 | 5 | 37.3 |
| 3au | 2-NC—C₆H₄ | 2470 | 541 | 18 | 11 | 30.1 |
| 3ax | 1-naphthyl-CH₂CH₂ | 7650 | 317 | 15 | 25 | 21.1 |
| 3ay | thiophen-2-yl- | 4300 | 236 | 9 | 3 | 26.2 |
| | CH₂CH₂ | | | | | |
| 3az | 3-(2,3-dihydro-benzo | 3800 | 438 | 6 | 4 | 73 |
| | [1,4]dioxin-6-yl) | | | | | |
| 3aw | N-Boc-piperidin-4-yl | 7640 | 257 | 7 | 2 | 36.7 |
| 3ba | pyridin-2-yl-CH₂ | 3010 | 421 | 12 | 5 | 35.0 |
| 3bb | pyridin-2-yl-CH₂CH₂ 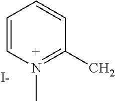 | 3095 | 443 | 10 | 14 | 44.3 |
| 3bc | 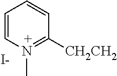 | 4320 | 403 | 8 | 7 | 50.3 |
| 3bd | 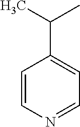 | 1850 | 376 | 6 | 8 | 62.6 |
| 3be | 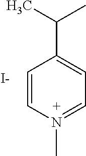 | 3270 | 258 | 10 | 8 | 25.8 |
| 3bg | 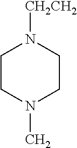 | 4100 | 301 | 16 | 12 | 18.8 |

### CA Inhibition.

An Applied Photophysics stopped-flow instrument has been used for assaying the CA catalysed CO₂ hydration activity.¹ Phenol red (at a concentration of 0.2 mM) has been used as indicator, working at the absorbance maximum of 557 nm, with 20 mM Hepes (pH 7.5) as buffer, and 20 mM Na₂SO₄ (for maintaining constant the ionic strength), following the initial rates of the CA-catalyzed CO₂ hydration reaction for a period of 10-100 s. The CO₂ concentrations ranged from 1.7 to 17 mM for the determination of the kinetic parameters and inhibition constants. For each inhibitor at least six traces of the initial 5-10% of the reaction have been used for determining the initial velocity. The uncatalyzed rates were determined in the same manner and subtracted from the total observed rates. Stock solutions of inhibitor (0.1 mM) were prepared in distilled-deionized water and dilutions up to 0.01 nM were done thereafter with distilled-deionized water Inhibitor and enzyme solutions were preincubated together for 15 min at room temperature in order to allow for the formation of the E-I complex. The inhibition constants were obtained by non-linear least-squares methods using PRISM 3, as reported earlier,^{1,2,3} and represent the mean from at least three different determinations. All enzymes were recombinant ones, obtained as reported earlier.^{2,3}
1. Khalifah, R. G. The carbon dioxide hydration activity of carbonic anhydrase. I. Stop-flow kinetic studies on the native human isoenzymes B and C. J. Biol. Chem. 1971, 246, 2561-2573.
2. Alterio, V.; Hilvo, M.; Di Fiore, A.; Supuran, C. T.; Pan, P.; Parkkila, S.; Scaloni, A.; Pastorek, J.; Pastorekova, S.; Pedone, C.; Scozzafava, A.; Monti, S. M.; De Simone, G. Crystal structure of the extracellular catalytic domain of the tumor-associated human carbonic anhydrase IX. Proc. Natl. Acad. Sci. USA, 2009, 106, 16233-16238.
3. a) Alterio, V.; Vitale, R. M.; Monti, S. M.; Pedone, C.; Scozzafava, A.; Cecchi, A.; De Simone, G.; Supuran, C. T. Carbonic anhydrase inhibitors: X-ray and molecular modeling study for the interaction of a fluorescent antitumor sulfonamide with isozyme II and IX. J. Am. Chem. Soc. 2006, 128, 8329-8335; b) Stiti, M.; Cecchi, A.: Rami, M.; Abdaoui, M.; Barragan-Montero, V.; Scozzafava, A.; Guari, Y.; Winum, J. Y.; Supuran, C. T. Carbonic anhydrase inhibitor coated gold nanoparticles selectively inhibit the tumor-associated isoform IX over the cytosolic ubiquitous isozymes I and II. J. Am. Chem. Soc. 2008, 130, 16130-16131.

### Example 3

### Preparation of Ureido-Sulfamides 7 and 8 with Potent CA IX/XII Inhibitory Activity

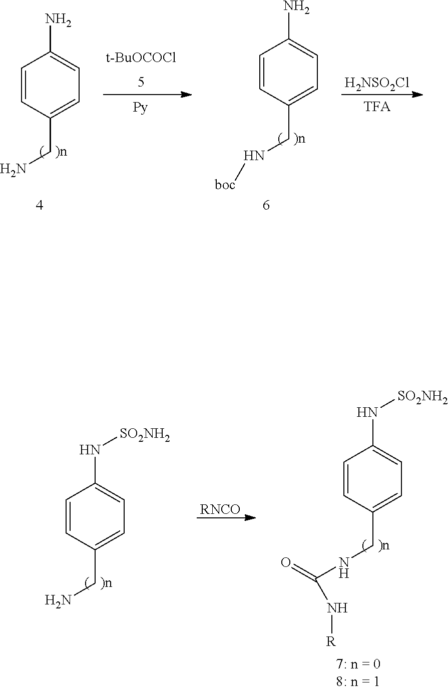

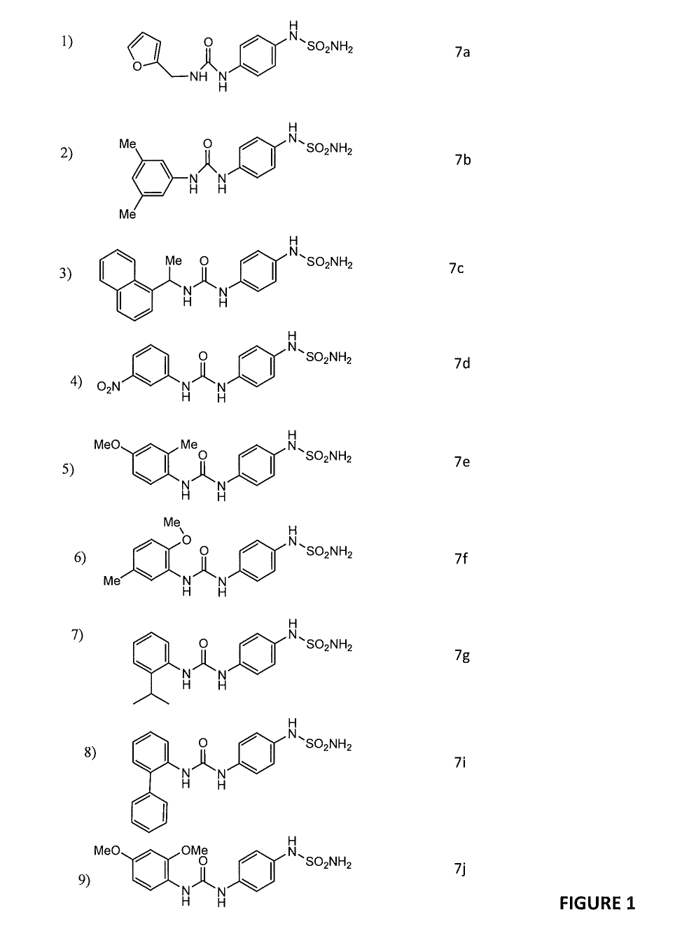

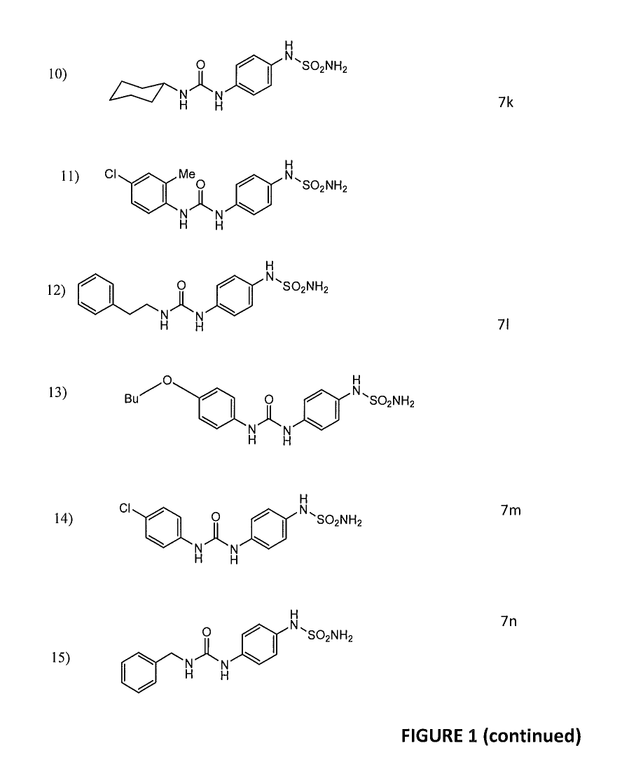

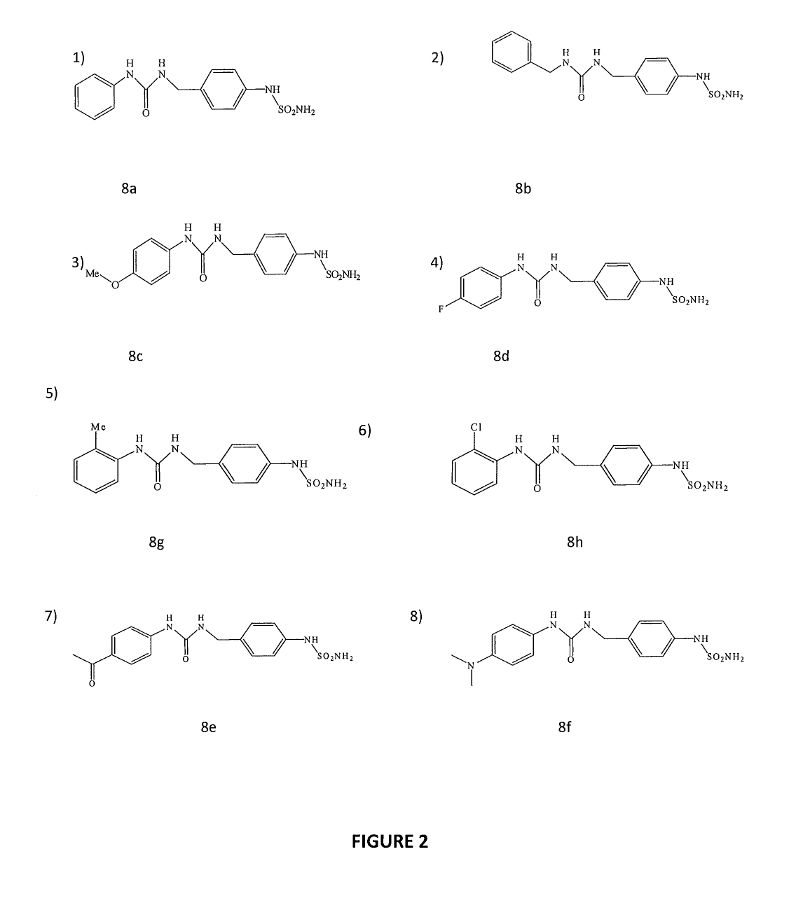

A series of ureido-sulfamides 7/8 were prepared, the structures of which are depicted in FIGS. 1 and 2, and as shown in the reaction scheme above. Starting from 1,4-phenylene-diamine 4, which has been monoprotected with the tertbutyl-oxycarbonyl (boc) moiety, by reaction with boc chloride 5, the key intermediates 6 have been obtained, which were not isolated. The one-pot preparation continued with the sulfamoylation of 6 (as described above for the preparation of sulfamates 3, Procedure B) and treatment with trifluoroacetic acid (TFA) which led to the deprotected amine. The sulfamides 7/8 were then prepared from the key intermediate, by reaction with alkyl/aryl isocyanates as described above for compounds 3, with an acceptable yield (of 45-63%). The analogues sulfamides 8, possessing an extra methylene moiety between thenureido and benzenesulfamide part of the molecule, were prepared similarly to 7, starting with 4-aminobenzylamine instead of 1,4-phenylenediamine.

### Example 4

### Inhibition Studies on Carbonic Anhydrases with Sulfamides 7

Inhibition studies were performed on carbonic anhydrases using the compounds prepared in accordance with Example 3. The inhibition constant (Ki) was determined for CA I, CA II, CA IX and CA XII using each of the prepared compounds 7a to 7p. This is set out in further detail below and the results are presented in Table 2.

TABLE 2
CA inhibition data with the sulfamides 7 and 8 described in the patent
7a-7p

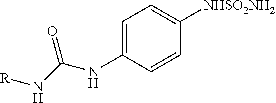

8a-8h

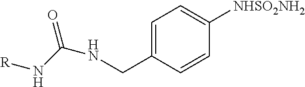

| | | | Ki (nM) | | | KiCAII/ |
|---|---|---|---|---|---|---|
| No | R | hCA I | hCA II | hCAIX | hCA XII | KiCAIX |
| 7a | furan-2-yl-CH₂ | 6800 | 345 | 19 | 12 | 18.1 |
| 7b | 3,5-Me₂C₆H₃ | 8400 | 674 | 10 | 5 | 67.4 |
| 7c | 1-naphthyl-CH(CH₃) | 7240 | 412 | 19 | 28 | 21.7 |
| 7d | 3-O₂N—C₆H₄ | 1980 | 423 | 16 | 8 | 26.4 |
| 7e | 2-Me-4-MeO—C₆H₃ | 3650 | 313 | 21 | 36 | 14.9 |
| 7f | 5-Me-2-MeO—C₆H₃ | 4300 | 436 | 40 | 26 | 10.9 |
| 7g | 2-iPr—C₆H₄ | 8340 | 673 | 98 | 54 | 6.8 |
| 7i | 2-Ph—C₆H₄ | 10500 | 894 | 145 | 240 | 6.1 |
| 7j | 2,5-(MeO)₂C₆H₃ | 4520 | 447 | 95 | 86 | 4.7 |
| 7k | cyclohexyl | 4000 | 354 | 121 | 98 | 2.9 |
| 71 | 2-Me-4-Cl—C₆H₃ | 3980 | 135 | 21 | 16 | 6.4 |
| 7m | 4-PhCH₂CH₂ | 2560 | 157 | 11 | 8 | 14.3 |
| 7n | 4-nBuO—C₆H₄ | 1540 | 312 | 16 | 9 | 19.5 |
| 7o | 4-Cl—C₆H₄ | 3760 | 315 | 43 | 25 | 7.3 |
| 7p | 4-PhCH₂— | 2500 | 146 | 10 | 8 | 14.6 |
| 8a | Ph | 3000 | 156 | 11 | 10 | 14.2 |
| 8b | 4-PhCH₂— | 2460 | 131 | 9 | 5 | 14.5 |
| 8c | 4-MeO—C₆H₄ | 2310 | 365 | 11 | 13 | 33.2 |
| 8d | 4-F—C₆H₄ | 2840 | 233 | 10 | 8 | 23.3 |
| 8e | 4-Ac—C₆H₄ | 3590 | 138 | 24 | 14 | 5.8 |
| 8f | 4-Me₂N—C₆H₄ | 1340 | 48 | 12 | 22 | 4.0 |
| 8g | 2-MeC₆H₄— | 3800 | 343 | 21 | 30 | 16.3 |
| 8h | 2-Cl—C₆H₄ | 1650 | 47 | 17 | 12 | 2.8 |

### Example 5

### Preparation of Thioureido-Sulfamates with Strong CAIX/XII Inhibitory Activity

### Materials and Methods

### Chemistry

Anhydrous solvents and all reagents were purchased from Sigma-Aldrich, Alfa Aesar and TCI. All reactions involving air- or moisture-sensitive compounds were performed under a nitrogen atmosphere using dried glassware and syringes techniques to transfer solutions. Nuclear magnetic resonance (¹H-NMR, ¹³C-NMR, DEPT-135, DEPT-90, HSQC, HMBC, ¹⁹F-NMR) spectra were recorded using a Bruker Advance III 400 MHz spectrometer in DMSO-d₆. Chemical shifts are reported in parts per million (ppm) and the coupling constants (J) are expressed in Hertz (Hz). Splitting patterns are designated as follows: s, singlet; d, doublet; sept, septet; t, triplet; q, quadruplet; m, multiplet; brs, broad singlet; dd, double of doubles, appt, aparent triplet, appq, aparent quartet. The assignment of exchangeable protons (OH and NH) was confirmed by the addition of D₂O. Analytical thin-layer chromatography (TLC) was carried out on Merck silica gel F-254 plates. Flash chromatography purifications were performed on Merck Silica gel 60 (230-400 mesh ASTM) as the stationary phase and ethylacetate/n-hexane were used as eluents. Melting points (mp) were carried out in open capillary tubes and are uncorrected.

### Abbreviation List

aq. aqueous
Ar—H aromatic protons
bs broad singlet
° C. temperature in degrees Centigrade

### DCC Dicyclohexylcarbodiimide

### DCM Dichloromethane

Decomp. Decomposition

### DMA Dimethylacetamide

DMAP N,N-dimethyl-4-amino pyridine

### DMF N,N-dimethylformamide

EDCl HCl N-(3-Dimethylaminopropyl)-N′-ethylcarbodiimide hydrochloride
eq equivalents
g gram(s)
h hour(s)

### HOBt 1-Hydroxybenzotriazole

### Hz Hertz

IR infra red
J coupling constant in Hz
uₘₐₓ wavenumber
min minutes
MHz mega Hertz
ppm parts per million
Pyr pyridine
r.t. room temperature
δ_{c} ¹³C chemical shift reported in ppm
δ_{F} ¹⁹F chemical shift reported in ppm
δ_{H} ¹H chemical shift reported in ppm
THF tetrahydrofuran
TLC thin layer chromatography

### General Procedure for the Synthesis of Isothiocyanates 9-10.¹

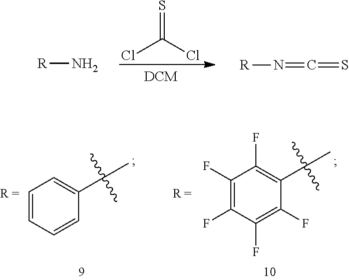

A 1.0 M solution of the corresponding amine (1.0 g, 1.0 eq) in dry DCM is cooled down to 0° C. and treated with thionyl chloride (3.0 eq). The orange solution was stirred at r.t. under a nitrogen atmosphere until starting material was consumed (TLC monitoring). Solvents were removed in vacuo to afford a residue that was used immediately without further purification.

### General Procedure for the Synthesis of Thioureas 11-14.²

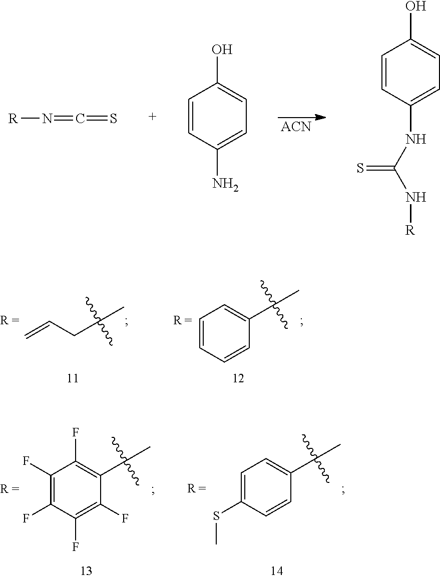

A 1.0 M solution of the appropriate isocyanate (1.0 eq) was dissolved in dry ACN and treated with 4-aminophenol (1.0 eq). The reaction mixtures were stirred vigorously at r.t. until starting material were consumed (TLC monitoring). The solids formed were separated by filtration, washed several times with water, dried under vacuo and purified by silica gel column chromatography eluting with 50% ethyl acetate/n-hexane to afford the title compounds 11-14.

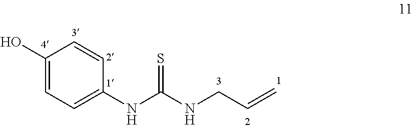

1-Allyl-3-(4-hydroxyphenyl)thiourea (11): yield 82% yield; silica gel TLC R_{f} 0.20 (Ethyl A acetate/n-hexane 50% v/v); δ_{H} (400 MHz, DMSO-d₆) 4.13 (2H, brs, 3-H₂), 5.12 (2H, m, 1-H₂), 5.90 (1H, m, 2-H), 6.75 (2H, d, J 8.8, 2×2′-H), 7.10 (2H, d, J 8.8, 2×3′-H), 7.10 (1H, brs, exchange with D₂O, NH-Allyl), 9.28 (1H, brs, exchange with D₂O, NH), 9.43 (1H, s, exchange with D₂O, OH); δ_{c} (100 MHz, DMSO-d₆) 181.8 (C═S), 155.9, 149.1, 141.6, 136.1, 130.8, 127.5, 47.1

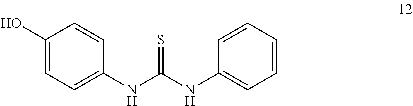 [SMILES: Oc1ccc(NC(=S)Nc2ccccc2)cc1]

1-(4-Hydroxyphenyl)-3-phenylthiourea (12): yield 79% yield; silica gel TLC R_{f} 0.25 (Ethyl A acetate/n-hexane 50% v/v); δ_{H} (400 MHz, DMSO-d₆) 6.75 (2H, d, J 8.8, Ar—H), 7.12-7.50 (7H, m, Ar—H), 9.40 (1H, brs, exchange with D₂O, OH), 9.54 (2H, brs, exchange with D₂O, 2×NH); δ_{c} (100 MHz, DMSO-d₆) 180.8 (C═S), 155.8, 140.6, 131.4, 129.3, 127.2, 125.2, 124.6, 115.9.

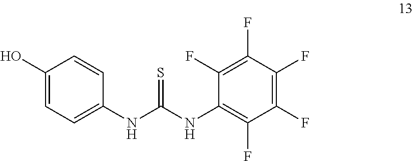 [SMILES: Oc1ccc(NC(=S)Nc2c(F)c(F)c(F)c(F)c2F)cc1]

1-(4-Hydroxyphenyl)-3-(perfluorophenyl)thiourea (13): yield 62% yield; silica gel TLC R_{f} 0.22 (Ethyl A acetate/n-hexane 40% v/v); δ_{H} (400 MHz, DMSO-d₆) 6.79 (2H, d, J 8.8, 2×2-H), 7.20 (2H, d, J 8.8, 2×3-H), 9.20 (1H, s, exchange with D₂O, Ar—H—NH), 9.55 (1H, s, exchange with D2O, OH), 10.12 (1H, s, exchange with D₂O, Ar—F—NH); δ_{c} (100 MHz, DMSO-d₆) 181.2 (C═S), 147.3, 145.2 (d, ¹J _{C—F} 239, C-2′), 142.0 (d, ¹J _{C—F} 241, C-4′), 140.1 (d, ¹J _{C—F} 251, C-3′), 139.4, 126.2, 123.2, 118.0 (m, ²J _{C—F} 22, ³J _{C—F} 8, C-1′); δ_{F} (376.5 MHz, DMSO-d₆) −144.8 (d, ³J _{F—F} 22.0, 2×F-2′), −157.30 (t, ³J _{F—F} 21.0, F-4′), −164.21 (t, ³J _{F—F} 22.0, 2×F-3′).

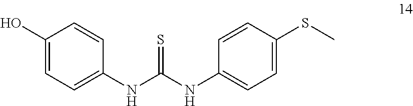 [SMILES: CSc1ccc(NC(=S)Nc2ccc(O)cc2)cc1]

1-(4-Hydroxyphenyl)-3-(4-(methylthio)phenyl)thiourea (14): yield 86% yield; silica gel TLC R_{f} 0.22 (Ethyl A acetate/n-hexane 50% v/v); δ_{H} (400 MHz, DMSO-d₆) 2.50 (3H, s, CH₃), 6.75 (2H, d, J 8.8, Ar—H), 7.19-7.44 (6H, m, Ar—H), 9.41 (1H, s, exchange with D₂O, OH), 9.53 (2H, brs, exchange with D₂O, 2×NH); δ_{c} (100 MHz, DMSO-d₆) 180.7 (C═S), 155.8, 137.9, 134.2, 131.4, 127.3, 127.2, 125.5, 116.0, 16.3.

### General Procedure for the Synthesis of Sulfamates 15-18

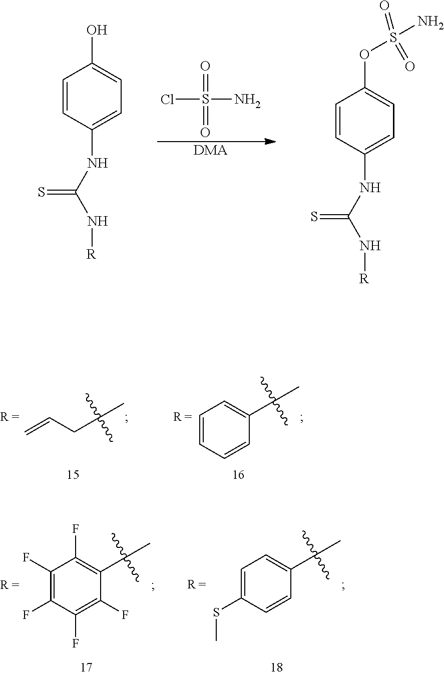

Freshly prepared chlorosulfanilamide was added to a 2.0 M solution of phenols 11-14 in dry DMA at 80° C. under a nitrogen atmosphere until starting material was consumed (TLC monitoring). For 18 the reaction was carried out at r.t. Then the solution was quenched with slush and extracted with ethyl acetate (3×20 ml). The combined organic layers were washed with H₂O (4×20 ml), brine (3×20 ml) dried over Na₂SO₄, filtered and concentrated under vacuo to give a sticky residue that was purified by silica gel column chromatography eluting with 50% ethyl acetate/n-hexane to afford the desired products.

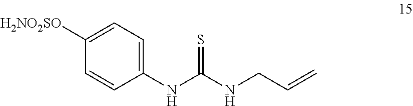

4-(3-Allylthioureido)phenyl sulfamate (15): yield 65% yield; silica gel TLC R_{f} 0.10 (Ethyl A acetate/n-hexane 50% v/v); δ_{H} (400 MHz, DMSO-d₆) 4.18 (2H, brs, NHCH₂), 5.21 (2H, m, CH═CH₂), 5.93 (1H, m, CH═CH₂), 7.25 (2H, d, J 8.8, 2×2′-H), 7.53 (2H, d, J 8.8, 2×3′-H), 7.92 (1H, brs, exchange with D₂O, NH-Allyl), 8.02 (2H, s, exchange with D₂O, SO₂NH₂), 9.65 (1H, s, exchange with D₂O, NH); δ_{c} (100 MHz, DMSO-d₆) 181.9 (C═S), 147.4, 138.7, 135.8, 129.0, 123.3, 116.9, 47.1.

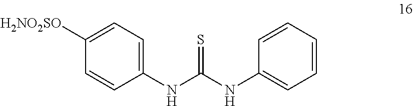

4-(3-Phenylthioureido)phenyl sulfamate (16): yield 70% yield; silica gel TLC R_{f} 0.15 (Ethyl A acetate/n-hexane 50% v/v); δ_{H} (400 MHz, DMSO-d₆) 7.18-7.60 (9H, m, Ar—H), 8.05 (2H, s, exchange with D₂O, SO₂NH₂), 9.88 (2H, s, exchange with D2O, 2×NH); δ_{c} (100 MHz, DMSO-d₆) 180.7 (C═S), 147.4, 140.3, 138.7, 129.4, 125.8, 125.5, 124.6, 123.0.

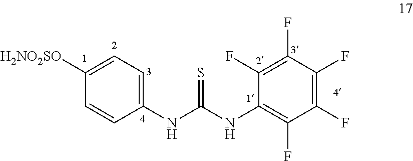

4-(3-(Perfluorophenyl)thioureido)phenyl sulfamate (17): yield 68% yield; silica gel TLC R_{f} 0.11 (Ethyl acetate/n-hexane 40% v/v); δ_{H} (400 MHz, DMSO-d₆) 7.31 (2H, d, J 8.8, 2×2-H), 7.58 (2H, d, J 8.8, 2×3-H), 8.09 (2H, s, exchange with D₂O, SO₂NH₂), 9.57 (1H, s, exchange with D₂O, Ar—H—NH), 10.44 (1H, s, exchange with D₂O, Ar—F—NH); δ_{c} (100 MHz, DMSO-d₆) 182.5 (C═S), 148.1, 144.9 (d, ¹J _{C—F} 240, C-2′), 140.6 (d, ¹J _{C—F} 242, C-4′), 138.2 (d, ¹J _{C—F} 249, C-3′), 138.1, 126.3, 123.4, 116.2 (m, ²J _{C—F} 24, ³J _{C—F} 10, C-1′); δ_{F} (376.5 MHz, DMSO-d₆) −145.1 (d, ³J _{F—F} 22.2, 2×F-2′), −156.72 (t, ³J _{F—F} 20.7, F-4′), −163.92 (t, ³J _{F—F} 22.2, 2×F-3′).

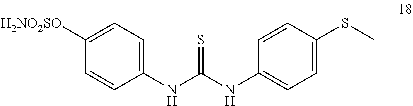

4-(3-(4-(Methylthio)phenyl)thioureido)phenyl sulfamate (18): yield 73% yield; silica gel TLC R_{f} 0.16 (Ethyl acetate/n-hexane 50% v/v); δ_{H} (400 MHz, DMSO-d₆) 2.51 (3H, s, SCH₃), 7.26-7.60 (8H, m, Ar—H), 8.05 (2H, s, exchange with D₂O, SO₂NH₂), 9.87 (2H, s, exchange with D2O, 2×NH); δ_{c} (100 MHz, DMSO-d₆) 181.6 (C═S), 147.4, 138.8, 137.5, 134.7, 127.4, 125.8, 125.4, 123.1, 38.4.

### Synthesis of 2-(3-hydroxy-6-oxo-6H-xanthen-9-yl)-5-(3-(4-hydroxyphenyl)thioureido)benzoic acid (19).²

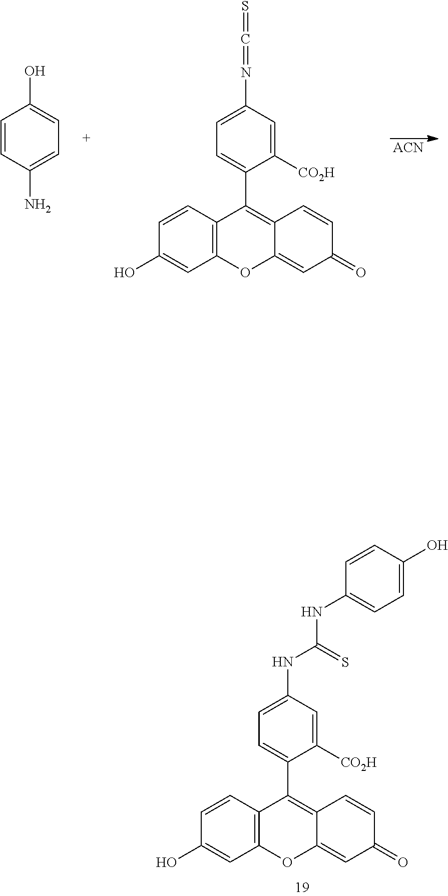 [SMILES: Nc1ccc(O)cc1 ; O=C(O)c1cc(N=C=S)ccc1-c1c2ccc(=O)cc-2oc2cc(O)ccc12 ; "ACN" ; O=C(O)c1cc(NC(=S)Nc2ccc(O)cc2)ccc1-c1c2ccc(=O)cc-2oc2cc(O)ccc12]

4-Aminophenol (0.1 g, 1.0 eq) was added to a suspension of fluoresceine isothiocyanate (0.36 g, 1.0 eq) in dry ACN (10 ml) and the reaction was stirred under a nitrogen atmosphere O.N. The solids were separated by filtration and the filtrate concentrated under vacuo. The residue was purified by silica gel column chromatography eluting with ethyl acetate to afford 19 as an orange solid in 57% yield.

2-(3-Hydroxy-6-oxo-6H-xanthen-9-yl)-5-(3-(4-hydroxyphenyl)thioureido)benzoic acid (19): silica gel TLC R_{f} 0.11 (Ethyl Acetate); δ_{H} (400 MHz, DMSO-d₆) 6.49 (1H, dd, J 7.5, 6′-H), 6.65 (4H, brs), 6.70 (2H, s), 6.80 (2H, d, J 8.8), 7.22 (2H, d, J 8.8), 8.83 (1H, d, J 7.5, 5′-H), 8.18 (1H, s, 2′-H), 9.49 (1H, s, exchange with D2O, 1-OH), 9.87 (1H, s, exchange with D₂O, NH), 9.95 (1H, s, exchange with D₂O, NH), 10.18 (1H, s, exchange with D₂O, OH); δ_{c} (100 MHz, DMSO-d₆) 180.9 (C═S), 169.6, 160.5, 156.2, 152.9, 148.6, 142.5, 131.9, 131.2, 131.0, 130.1, 127.4, 127.38, 125.0, 118.7, 116.7, 116.6, 116.2, 113.7, 110.7, 103.3, 84.1

### Synthesis of 2-(3-hydroxy-6-oxo-6H-xanthen-9-yl)-5-(3-(4-(sulfamoyloxy)phenyl)thioureido)benzoic acid (20)

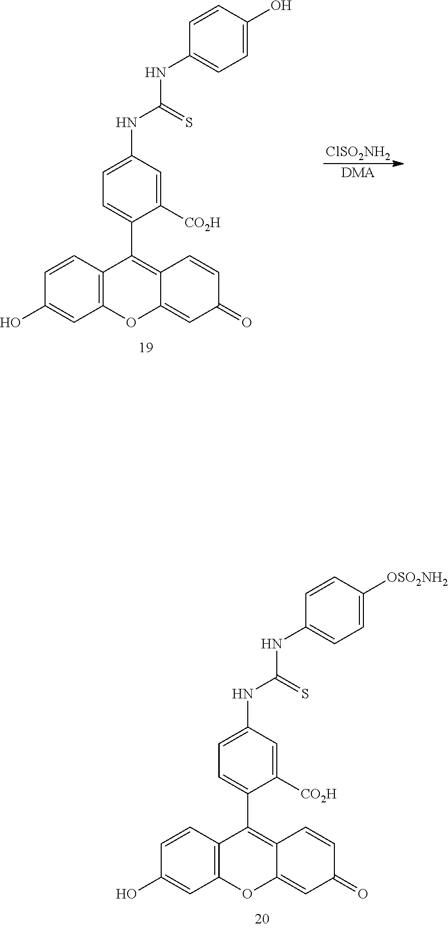

2-(3-Hydroxy-6-oxo-6H-xanthen-9-yl)-5-(3-(4-(sulfamoyloxy)phenyl)thioureido)benzoic acid (20): yield 56% yield; silica gel TLC R_{f} 0.07 (Ethyl acetate); δ_{H} (400 MHz, DMSO-d₆) 7.06-7.38 (8H, m), 7.40 (2H, s), 7.62 (2H, d, J 8.8), 7.93 (1H, d, J 7.5), 8.10 (2H, s, exchange with D₂O, SO₂NH₂), 8.26 (4H, s), 8.32 (1H, s), 10.20 (1H, s, exchange with D₂O, NH), 10.31 (1H, s, exchange with D₂O, NH); δ_{c} (100 MHz, DMSO-d₆) 180.8 (C═S), 169.1, 162.0, 152.5, 151.7, 148.3, 147.8, 145.8, 142.7, 138.9, 138.3, 131.8, 130.5, 127.5, 126.6, 126.1, 125.0, 123.7, 123.3, 119.8, 119.7, 118.8, 117.9, 111.5, 81.6.

### REFERENCES

1) Mays, Jared, Rae and Rajski, Scott, R, Patent WO 2008/008954 A2,
2) Fabio Pacchiano, Mayank Aggarwa, Balendu Sankara Avvaru, Arthur H. Robbins, Andrea Scozzafava, Robert McKenna and Claudiu T. Supuran, Selective hydrophobic pocket binding observed within the carbonic anhydrase II active site accommodate different 4-substituted-ureidobenzenesulfonamides and correlate to inhibitor potency, Chem. Commun., 2010, 46, 8371-8373.

### Example 6

### Inhibition Studies on Carbonic Anhydrases with Sulfamates 15 to 18 and 20

Inhibition studies were performed on carbonic anhydrases using the compounds prepared in accordance with Example 5. The inhibition constant (Ki) was determined for CAI, CAII, CAIX and CAXII. The results are presented in Table 3.

TABLE 3
CA inhibition data with compounds 15, 16, 17, 18 and 20
(stopped flow assay)

| Com- | Ki (nM) | | | | |
|---|---|---|---|---|---|
| pound | hCA I | hCA II | hCA IX | hCA XII | Ki CA II/Ki CA IX |
| 7 | 6530 | 541 | 12 | 15 | 45.1 |
| 8 | 6725 | 347 | 19 | 6 | 18.3 |
| 9 | 5418 | 613 | 9 | 5 | 68.1 |
| 10 | 3459 | 486 | 10 | 10 | 48.6 |
| 12 | 7635 | 235 | 14 | 12 | 16.8 |

### Example 7

### Anti-Tumour Activity of Selected Ureido-Sulfamate

Compound 3p was selected for further investigation. This compound was the ureido-sulfamate 4-[3,5-dimethylphenyl)ureido]phenyl sulfamate, which had a Ki for CA IX of 2 nM and a Ki for CA XII of 7 nM. This compound also had a selectivity ratio (Ki II/Ki IX) of 78.

A mouse xenograft model was chosen to assess the in vivo activity of this inhibitor on HT29 colon carcinoma cells which had been subcutaneously injected into mice to form a xenograft. The experimental details are set out in Table 4

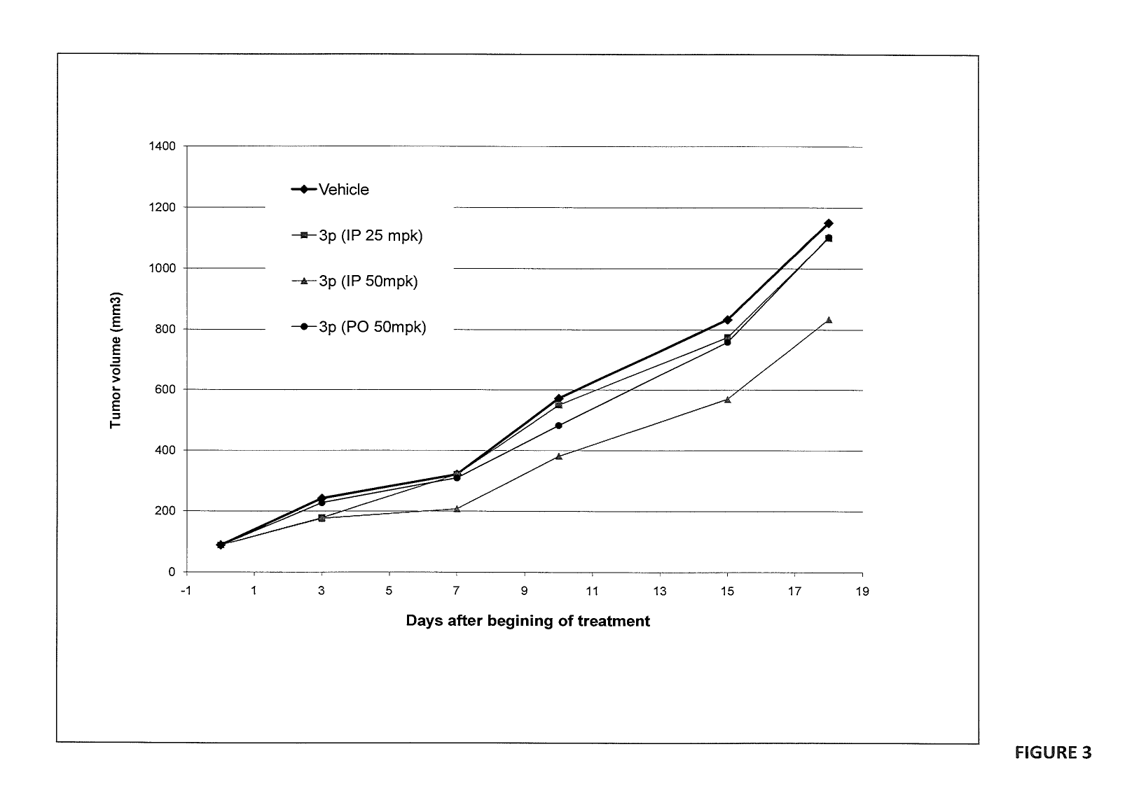

The results are shown in FIG. 3 from which it may be inferred that a significant reduction in the volume of the tumour is observed when comparing subcutaneous injection of carbonic anhydrase inhibitor with control. As around 30% of the cells in the tumor are hypoxic, and as it can be seen, at the highest dosage of CA inhibitor, a reduction of the tumor growth of 30% has been achieved, it can be concluded that all the cancer cells expressing CA IX have been killed by inhibiting the enzyme with the CA IX-sulfamate inhibitor from the invention.

Taken together, the results indicate that the CA inhibitors of the present invention are potent inhibitors of CA IX and CA XII and demonstrate selectivity for inhibition of CA IX or CA XII over their intracellular isozyme counterparts. Activity of the inhibitors in vivo in the reduction of tumour size has also been demonstrated.

TABLE 4 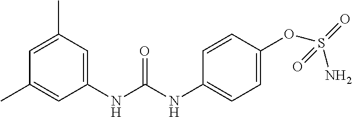 [SMILES: Cc1cc(C)cc(NC(=O)Nc2ccc(OS(N)(=O)=O)cc2)c1]
Ki = 2 nM (CA IX) Ki = 7 nM (CA XII)
Nude female CD1 mice, 7 week old (Charles River France) were
injected subcutaneously with 4 millions of HT29 colon carcinoma cells
in physiologic suspension (200 ul). 14 days after injection of cells;
animals began treatment (5 ml/kg). The treatment continued for 18 days.
Animals were divided into four separate groups (10 animals per group):

| Compound | Dose & Regime | Via | duration |
|---|---|---|---|
| Vehicle | QD 5 days a week | I.P. | 3 weeks |
| (10%DMS0/45% | | | |
| PEG400/45%Water) | | | |
| 3p | 25 mpk QD 5 days a week | I.P. | 3 weeks |
| 3p | 50 mpk QD 5 days a week | I.P. | 3 weeks |
| 3p | 50 mpk QD 5 days a week | PO | 3 weeks |

### Example 8

### Anti-Metastatic Activity of CAIX Inhibitors

CAIX inhibitor 3p was assessed for its effect on in vitro and in vivo models of tumour metastasis.

An in vivo experiment was undertaken as follows:

16 mice were implanted with 0.1 ml of a 5×10⁷/ml suspension of MDA231-EGFP cells into the mammary fat pad of anaesthetized mice. The cells were prepared in a 1:1 mix of Matrigel: serum-free RMPI.

Mouse condition was monitored daily and tumour volumes recorded at least 3× per week.

Once tumours reached approximately 100 mm³, they were randomised in to the following treatment groups:

Group 1: 8 mice implanted with MDA231-EGFP cells received vehicle administered in a “5 days on, 2 days” off schedule (ie Mon-Fri dosing each week).

Group 2: 8 mice implanted with MDA231-EGFP cells received CA-IX inhibitor S4 administered at 10 mg/kg dose in a “5 days on, 2 days” off schedule (ie Mon-Fri dosing each week).

Treatment was continued until primary tumours reached a designated endpoint volume of approx 1000 mm³.

Mouse health and condition was monitored throughout and weights recorded up to 3 times per week.

Before sacrifice Pimonidazole at 0.2 ml of 10 mg/ml solution i/p (2 h before) and 0.1 ml of a 6 mg/ml Hoechst solution (iv) (1 minute before) were administered. Tumour and lungs were rapidly removed for processing.

Clonogens were assessed in lungs using a clonogenic assay. About a quarter of the total lung tissue was taken and weighed. The tissue was cross chopped with scissors and a scalpel in a petri dish. 5 ml of RPMI medium supplemented with enzymes was added to the petri dish and this was incubated for 40 minutes on a shaker at 37° C. The RPMI serum free medium (47 ml) was formulated with 66 mg collagenase, 18.94 mg trypsin and 1 mg DNase. After incubation, 5 ml RPMI with 10% FCS was added to neutralize the enzymes. The sample was pipetted up and down to desegregate undigested parts and centrifuged for 3 minutes at 1500 rpm followed by resuspension in 4 ml BPS. A dilution range of cell suspensions was made in a 6-well plate and cells were left to grow for 5 to 7 days without changing the medium. Clones were then stained with bromophenol blue and counted, calculating the number per mg of tissue following correction for the dilution.

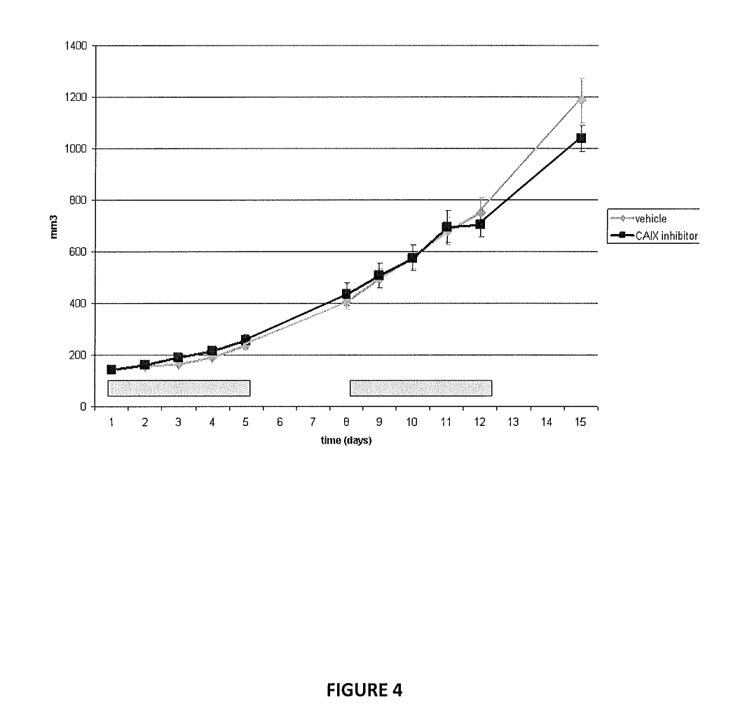

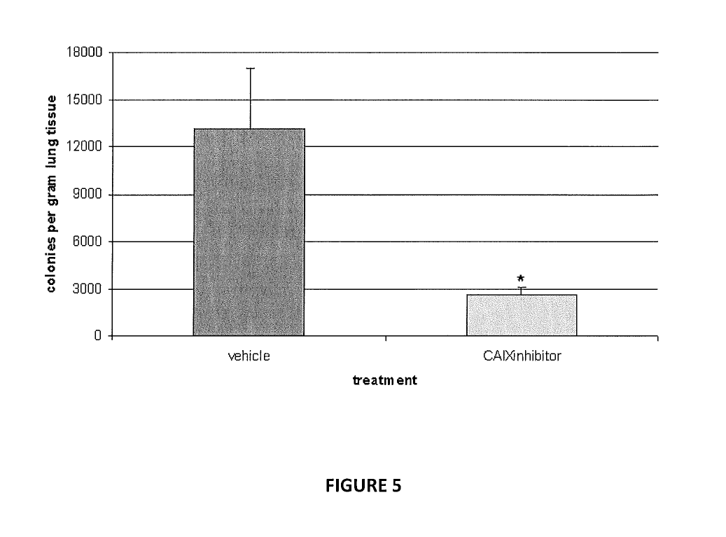

The results of this experiment are shown in FIGS. 4 and 5. FIG. 4 shows a graph of tumour volume against time comparing tumours from the vehicle treated mice against those treated with the CAIX inhibitor. It will be apparent from this Figure that the inhibitor had little or no effect on the growth of the orthotopic MDA-231 tumours, as assessed by tumour volume.

FIG. 5 shows a comparison of the number of colonies per gram of lung tissue as a measure of the number of metastatic MDA-231 clonogens in the treated mice. Comparing vehicle-treated mice with those treated with the CAIX inhibitor, it is clear that the CAIX inhibitor treated mice had significantly fewer metastatic clonogens suggesting that CAIX inhibitors may be potent anti-metastatic agents.

In the in vitro experiment a cell migration assay was performed to assess the effect of CAIX inhibitor 3p on in vitro wound closure.

A cell migration assay was performed as follows. A coverslip was placed in a 3 cm dish and 0.5×10⁶ cells (in 10% FCS-RPMI) was seeded, per 3 cm dish. The cells were left to grow for 24 hours so as to obtain a confluent layer of cells on the coverslip and a scratch was made with a pipette tip (p200) and loosely attached cells were washed off. The medium was replaced with a low serum (0.2% serum) medium to reduce the level of proliferation.

Inhibitor or vehicle was added and left for 0, 4, 8 or 24 hours. Inhibitor was applied at a working concentration of 33 μM and inhibitors were provided from a stock solution of 100 mM in DMSO. Cells were fixed in buffered formalin and scratches were imaged with ImageJ and the extent of wound closure was calculated. The cells used in the assay were MDA-231 GFP cells.

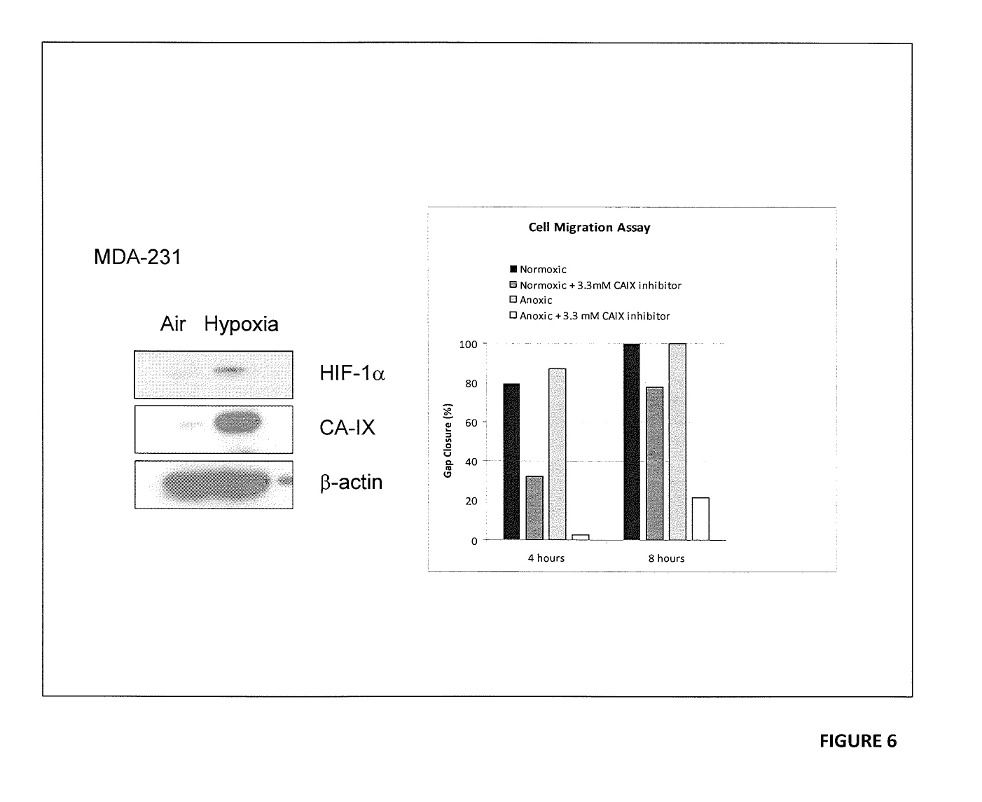

The results are shown in FIG. 6 on the right hand side. Gap closure (%) is plotted at 4 and 8 hours for vehicle-treated and inhibitor-treated cells under normoxic and anoxic conditions respectively. It is apparent from the results that the presence of CA-IX inhibitor significantly inhibits cell migration, thereby resulting in a very low percentage gap closure as compared with control.

The ability for MDA231 cells to express HIF and CA-IX under normoxic and anoxic conditions was assessed. MDA231 cells were grown in a standard cell culture incubator under normoxic/anoxic conditions for 24 hours. Cells were isolated from petri dishes and cell lysates prepared. The cell lysates were prepared by lysing the cells in TNN buffer supplemented with inhibitors to protect the proteins from degradation during the isolation. TNN buffer contained Tris-HCl, NaCL, EDTA, NP40 (supplemented on the day of use with DTT), PMSF, sodium orthovanadate, NaF, β-glycerol phosphate, NaPPi and protease inhibitor cocktail. The level of protein in the lysate was measured on a spectrophotometer against a concentration range of albumen. 50 micro grams of protein/lysate was loaded on a polyacrylamide gel to separate proteins according to their molecular weight. Proteins were transferred from the gel onto a nitrocellulose membrane. The membrane was incubated overnight with antibodies specific to CA-IX, HIF or β actin. The final detection was done by exposure of the membranes to a CL-XPosure film which is an X ray film to capture the emission of light after exposure of the membrane to enhanced chemiluminescence (using a horseradish peroxidase and hydrogen peroxide catalysed oxidation of luminol.

The results are shown in FIG. 6 on the left hand side, which shows images of the antibody-treated gel bands. The bands shown in the image reflect how much protein there is present in the cells. It is apparent that, under normal conditions in air no HIF or CA-IX is expressed.

This is because HIF is only expressed under low oxygen conditions and HIF is required to regulate CA-IX expression. Under hypoxic conditions both HIF and CA-IX are expressed. The β actin is present as a control and is expressed irrespective of the oxygenation of the cells.

### Anti-Metastatic Activity of CA IX Inhibitors (2)

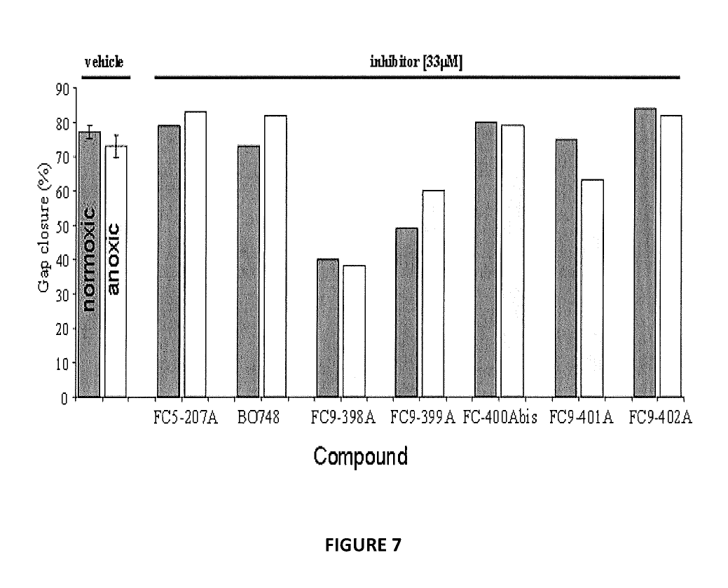

A series of 11 compounds were assayed in scratch wound assays to evaluate the effect of CA-IX inhibition on cellular migration in air and hypoxia. As a preliminary screen, compounds were tested against the MDA231 cell line at a concentration of 33 μM. This cell line is highly migratory in cell culture and shows robust induction of CA-IX in hypoxia (FIG. 6). Cells were then cultured in air or hypoxia and migration analysed 24 h later relative to vehicle treated controls. 7 compounds showed either little activity, or equivalent inhibitory effects in both air and hypoxia (FIG. 7). FIG. 7 shows that some of the compound panel showed little activity in the migration assay, or were equally able to prevent migration in both aerobic and hypoxic conditions (eg FC9-398A, FC9-399A, C-400Abis, FC9-401A and FC9-402A; corresponding respectively to compounds 3m, 3n, 3a and 3F).

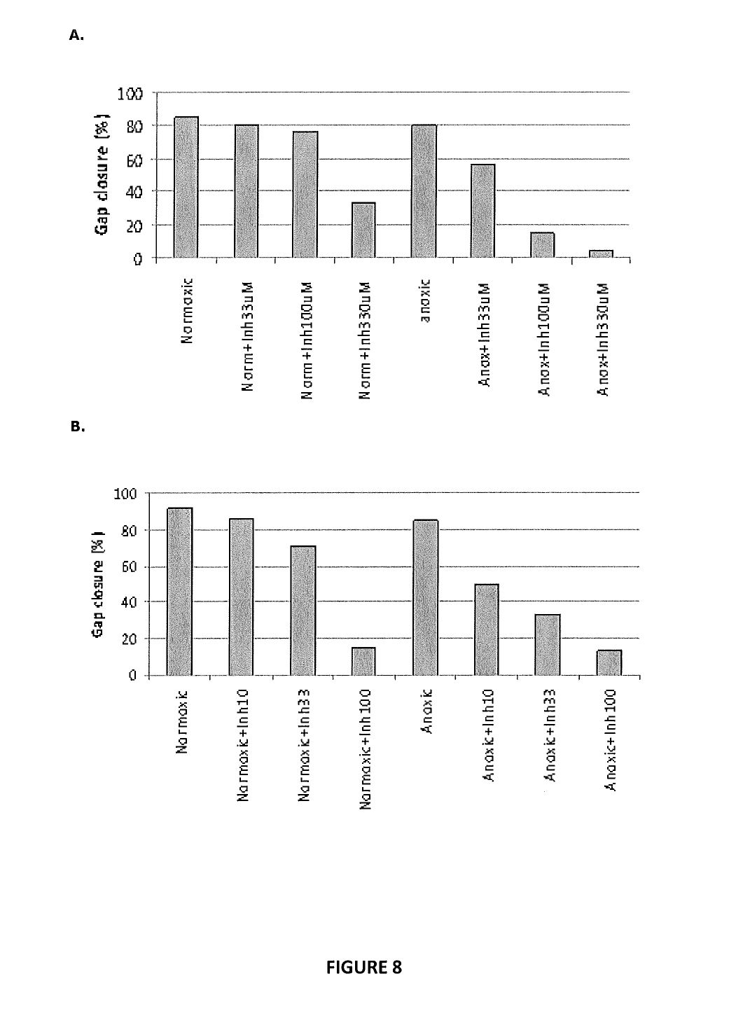

4 compounds (FC9-396A, FC-397A, FC-403A and S4; corresponding respectively to compounds 3i, 3k, 3h and 3p) showed inhibitory effects against hypoxic cell migration at concentrations that had minimal effects in air. These were deemed as apparent “hits” in this assay screen. FIG. 8 shows the dose response of FC-397A (A) and S4 (B). Inhibition by FC-397A was confirmed in two other cell lines (WRO; thyroid; HT1080; fibrosarcoma). Concentrations given are in μM.

These data support the contention that CA-IX inhibition can control spontaneous metastatic dissemination/growth from primary tumours.

### Example 9

### Enhancement of Chemotherapeutic and Radiotherapeutic effects of CA-IX Inhibitors

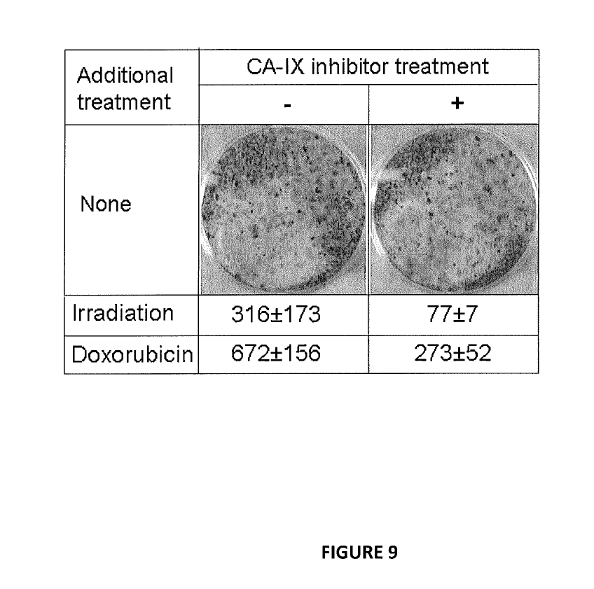

Further preliminary studies have been undertaken to evaluate the ability of the CA-IX inhibitor, S4 (corresponding to compound 3p from Example 1) to enhance the effect of chemo or radiotherapy. These studies were undertaken using tumour cells grown into 3-D spheroid cultures that have a natural oxygen gradient. Spheroids of FaDU head and neck cells were generated and treated for 24 h with S4 or vehicle alone; doxorubicin alone or in combination with S4; and S4 or vehicle plus 10 Gy radiotherapy given at the end of the 24 h exposure. Spheroids were treated with trypsin to generate single cell suspensions and plated at various dilutions. Clonogenecity was then recorded per spheroid. S4 alone little effect on growth of cells isolated from the spheroids. However coincident CA-IX inhibition and either doxorubicin or radiation treatment reduced colongenic survival compared with either agent alone (FIG. 9).

FIG. 9 shows spheroids derived from FaDU cells treated with the CA-IX inhibitor S4 alone (33 μM) or in combination with radiation (10 Gy) or doxorubicin (10 μM). The CA-IX inhibitor alone had little effect on cell growth (compare plates labelled “None”), but when combined with doxorubicin or radiotherapy there was a significant reduction in colony formation per spheroid (values given ±SEM).

These data suggest that CA-IX inhibition can improve the cytotoxic effect of radiotherapy or doxorubicin treatment in 3-D model systems where treatment resistance is linked to the presence of hypoxic cells.

### Method Details

### Spheroid Experiments:

Spheroids were generated by the “liquid overlay technique”. Fadu cells (1×10⁴ per well) were grown in a 96-wells plate of which were coated with agarose (1.5%). The coating prevents the cells from forming monolayers and gives rise of spheroid growth.

The spheroids were grown in a CO₂-incubator for 5-7 days. Thereafter, the spheroids were collected and grown in a spinner flask for an additional 5-7 days until they reached a diameter of ˜500-750 μm (10%-0.1% O₂ gradient in spheroid).

Spheroids were pooled for the following treatments:
1. Vehicle for 24 hours
2. CA9 inhibitor (3 μM) for 24 h
3. Doxorubicin (10 μM) for 24 h
4. Vehicle for 24 hours followed by 10 Gy radiotherapy
5. CA9 inhibitor (33 μM) for 24 h by 10 Gy radiotherapy

Directly thereafter, spheroids were digested to single cells using trypsin/EDTA and the cells seeded at a range of concentration in culture plates (clonogenicity assay).

After about a week the cells were stained with methylene blue and the number of colonies counted.

### Example 10

### Studies on Use of Fluorescent CA IX Inhibitors as Imaging/Diagnosis Agents

Compound 20, the fluorescent derivative FC11-489A bis from Example 5 possesses a fluorescent label and was used to study the effectiveness of CA IX inhibitors of the present invention in imaging/diagnosis applications.

This compound was tested in a final concentration of 100 μM (in 0.5% DMSO concentration). A stock solution of 1 mM was prepared containing 5% DMSO supplemented with DMEM cell culture growth medium and added on the cells in a 1/10 dilution. For the tests, human colorectal HT-29 adenocarcinoma cells were used harbouring a shRNA against CA IX (94/1) or a scrambled control (EV/2) [is there a public source of this cell line?].

### 10.1 Characterisation of Cell Lines

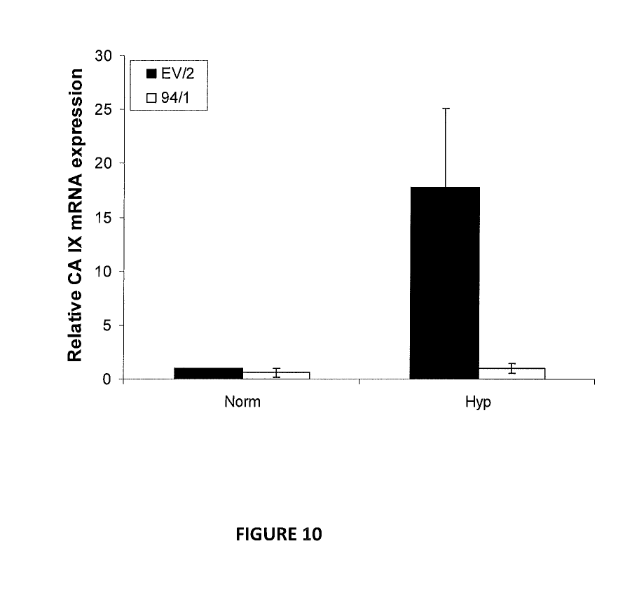

Exponentially growing cells were cultivated in Dulbecco's modified Eagle's medium supplemented with 10% fetal bovine serum. The cells were investigated by Western blotting and qRT-PCR for their CA IX expression levels under normoxia, hypoxia 0.2% or upon reoxygenation (qRT-PCR only under normoxia and hypoxia 0.2%). FIG. 10 shows CA IX mRNA expression levels upon 24 h exposure to hypoxia 0.2% (Hyp) in CA IX expressing (EV/2) and CA IX knock down (94/1) cell line. Normoxia (Norm) exposure was used as control. CA IX mRNA expression levels were significantly increased in the EV/2 CA IX expressing cell line upon hypoxia exposure (FIG. 10), while the 94/1 CA IX knock down cell line did not demonstrate an induction.

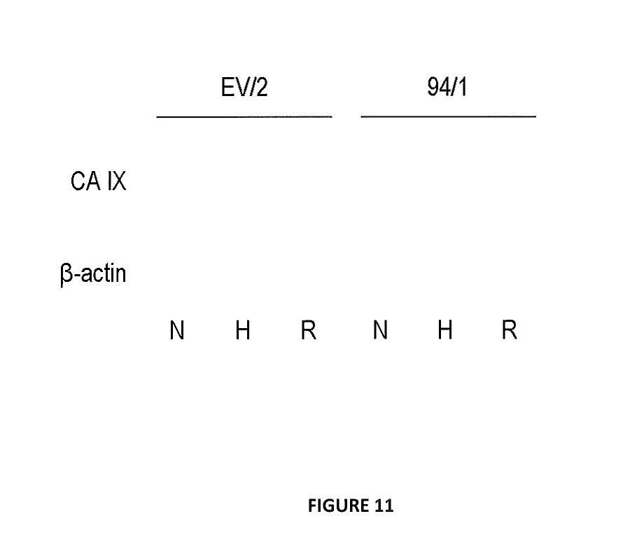

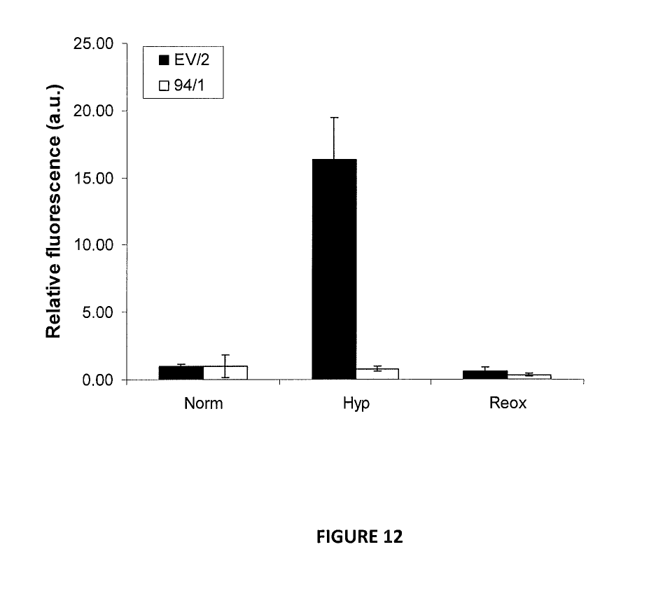

FIG. 11 shows CA IX protein expression levels upon 24 h exposure to normoxia (N), hypoxia 0.2% (H) and upon reoxygenation (R=24 h 0.2% followed by 1 h 21% oxygen) in CA IX expressing (EV/2) and CA IX knock down (94/1) cell line. β-actin was used as loading control. CA IX protein expression (FIG. 12) was found in the EV/2 cells and levels were elevated upon hypoxia 0.2%, which remained high upon reoxygenation (1 h) conditions. On the other hand, little to no CA IX protein expression was found in the 94/1 cells (>90% knock down) without any upregulation upon hypoxia or reoxygenation conditions.

### 10.2 FC11-489A Bis Binding

### 10.2.1 Plate Reader Experiment

EV/2 and 94/1 cells were plated at a density of 100.000 per well (Corning 24-well plates) a day before the start of the experiment and transfer to a hypoxic culture chamber (MACS VA500 micro-aerophilic workstation, Don Whitley Scientific, Shipley, UK). The atmosphere in the chamber consisted of 0.2% O₂, 5% CO₂ and residual N₂. Normoxic wells were incubated in parallel in air with 5% CO₂. Reoxygenation conditions were obtained by transferring plates after 24 h hypoxia exposure to air conditions for an additional hour. Cells were incubated with FC11-489A bis the last 30 min of each exposure. Control conditions were obtained by addition of 0.5% DMSO supplemented with medium. After incubation, cells were rinsed twice with PBS to remove unbound FC11-489A bis and fixed in freshly prepared 2% paraformaldehyde on ice. Plates were placed in a BMG microplate reader FLUOstare Omega using following protocol: Fluorescence intensity—Well scanning using 5×5 scan matrix in a diameter of 10 mm with 10 flashes per scan point, a gain of 1000 and 355 nm-460 nm filter settings. Fluorescence intensity data were corrected for both background signals (cells without FC11-489A bis) and normalized to the signal intensity of cells incubated with FC11-489A bis under normoxia.

A significant higher binding (P=0.004) of FC11-489A bis was demonstrated at EV/2 CA IX expressing cells exposed to hypoxia for 24 h, compared with their normoxic counterparts (FIG. 12), corresponding with elevated CA IX protein expression (FIG. 11). FIG. 12 shows quantitative fluorescence Omega plate reader analysis of FC11-489A bis binding to EV/2 CA IX expressing and 94/1 CA IX knock down cells treated under the respective conditions. Data demonstrate the fold accumulation compared with FC11-489A bis treated normoxic cells and represent the mean+/−SEM of four independent experiments. Upon reoxygenation, binding was dramatically reduced (P=0.002) compared with hypoxic conditions and was not statistically different (P=0.325) with binding under normoxia. No significant binding was found at 94/1 CA IX knock down cells, corresponding with having no CA IX protein expression in these cells (FIG. 10). Binding between EV/2 and 94/1 cells was only significantly different (P<0.001) upon hypoxia exposure.

### 10.2.2 Flow Cytometer Experiment

EV/2 and 94/1 cells were plated at a density of 0.5×10e6 per 6 cm dish (Corning) a day before the start of the experiment and transfer to a hypoxic culture chamber (MACS VA500 micro-aerophilic workstation, Don Whitley Scientific, Shipley, UK). The atmosphere in the chamber consisted of 0.2% O₂, 5% CO₂ and residual N₂. Normoxic dishes were incubated in parallel in air with 5% CO₂. Reoxygenation conditions were obtained by transferring dishes after 24 h hypoxia exposure to air conditions for an additional hour. Cells were incubated with FC11-489A bis the last 30 min of each exposure. Control conditions were obtained by addition of 0.5% DMSO supplemented with medium. After incubation, cells were rinsed twice with PBS to remove unbound FC11-489A bis, scraped and fixed in freshly prepared 2% paraformaldehyde on ice. Single suspensions were obtained by passing cells through 70 μm nylon cell strainers (BD Biosciences). Mean fluorescence intensity was analyzed using a FACSort flow cytometer (BD Biosciences) using FIT-C filter settings. Data were corrected for both background signals (cells without FC11-489A bis) and normalized to the signal intensity of cells incubated with FC11-489A bis under normoxia.

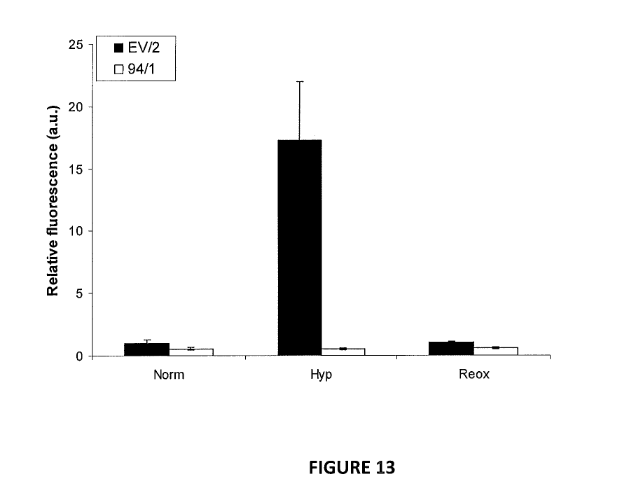

The FACS results confirmed the data obtained previously with the plate reader. FIG. 13 shows quantitative FACS analysis of FC11-489A bis binding to EV/2 CA IX expressing and 94/1 CA IX knock down cells treated under the respective conditions. Data demonstrate the fold accumulation compared with FC11-489A bis treated normoxic cells and represent the mean+/−SEM of four independent experiments. A significant higher binding (P=0.004) of FC11-489A bis was demonstrated at EV/2 CA IX expressing cells exposed to hypoxia for 24 h, compared with their normoxic counterparts (FIG. 13), corresponding with elevated CA IX protein expression (FIG. 11). Upon reoxygenation, binding was dramatically reduced (P=0.0016) compared with hypoxic conditions and was not statistically different (P=0.325) with binding under normoxia. No significant binding was found at 94/1 CA IX knock down cells, corresponding with having no CA IX protein expression in these cells (FIG. 12). Binding at EV/2 cells was significantly higher then at 94/1 cells for all experimental conditions (N: P=0.0043; H: P=0.0003; R: P=0.0022).

### 10.2.3 Fluorescence Staining Experiment

EV/2 and 94/1 cells were grown (at a density of 70000 cells) on glass coverslips a day before the start of the experiment and transfer to a hypoxic culture chamber (MACS VA500 micro-aerophilic workstation, Don Whitley Scientific, Shipley, UK). The atmosphere in the chamber consisted of 0.2% O₂, 5% CO₂ and residual N₂. Normoxic slides were incubated in parallel in air with 5% CO₂. Reoxygenation conditions were obtained by transferring the coverslips after 24 h hypoxia exposure to air conditions for an additional hour. Cells were incubated with FC11-489A bis the last 30 min of each exposure. Control conditions were obtained by addition of 0.5% DMSO supplemented with medium. At the end of the experiment, slides were rinsed twice with PBS to remove unbound FC11-489A bis and cells were fixed in freshly prepared 2% paraformaldehyde. Cells were mounted onto slides with Fluorescence Mounting Medium (DAKO) and analyzed with a Zeiss Axioskop fluorescence microscope using FIT-C filter settings.

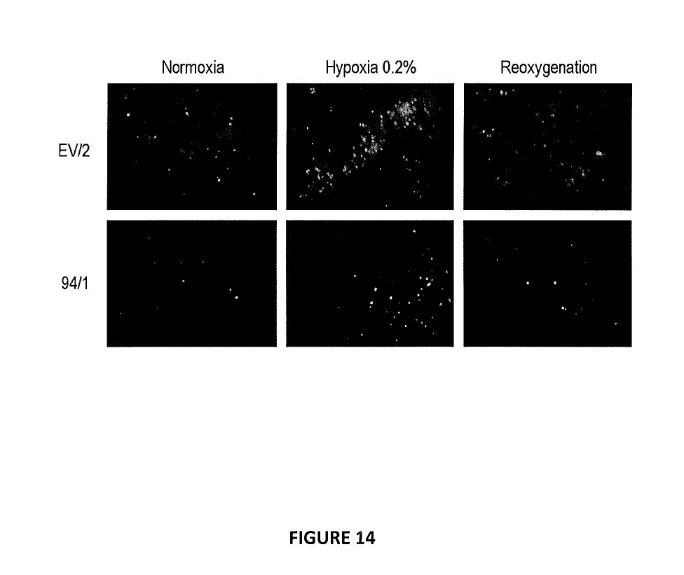

Immunofluorescence analysis demonstrated higher binding of FC11-489A bis at EV/2 CA IX expressing cells exposed to hypoxia 0.2% (FIG. 14). Binding was reduced upon reoxygenation to levels similar as normoxia exposure. Binding at 94/1 CA IX knock down cells was lower for all experimental conditions compared to the EV/2 cells.

Each staining picture was loaded into Image) analysis software (Image J 1.38x NIH USA, http://rsb.info.nih.gov/ij/Java 1.5.0_{—}17), converted to 8-bit images, thresholded to exclude areas without cells and all remaining pixels were analyzed for their intensity. Data were corrected for both background signals (cells without FC11-489A bis) and normalized to the signal intensity of cells incubated with FC11-489A bis under normoxia.

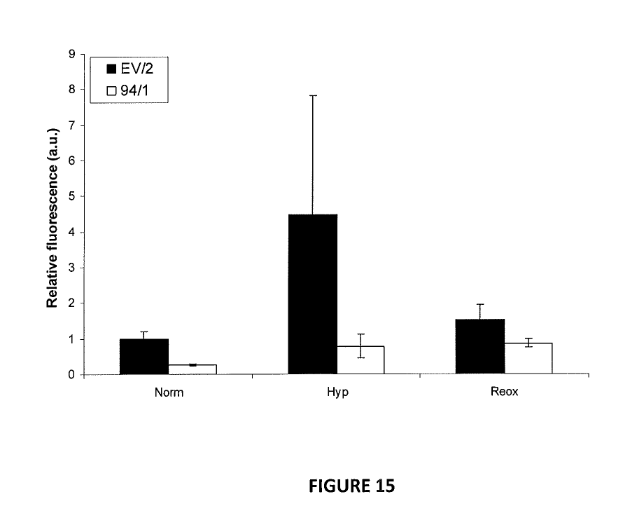

FIG. 15 shows pixel quantification of immunofluorescence staining of FC11-489A bis binding to EV/2 CA IX expressing and 94/1 CA IX knock down cells treated under the respective conditions. For each oxygen condition, for at least 350 pixels fluorescence intensity was analyzed.

A significant higher binding (P<0.001) of FC11-489A bis was demonstrated at EV/2 CA IX expressing cells exposed to hypoxia for 24 h, compared with their normoxic counterparts (FIG. 15), corresponding with elevated CA IX, protein expression (FIG. 11). Upon reoxygenation, binding was dramatically reduced (P=0.017) compared with hypoxic conditions and was not statistically different (P=0.134) with binding under normoxia. No significant binding was found at 94/1 CA IX knock down cells, corresponding with having no CA IX protein expression in these cells (FIG. 11).

### 10.2.3 Conclusion

HT-29 EV/2 CA IX expressing cells exposed to hypoxia demonstrated a strong CA IX upregulation both on mRNA and protein levels. Upon reoxygenation, CA IX protein expression levels stayed elevated, in agreement with the known half-life of 38 h in reoxygenated cells. HT-29 94/1 CA IX knock down cells demonstrated >90% reduction in CA IX expression and no upregulation was demonstrated upon hypoxia and reoxygenation conditions.

FC11-489A bis binding was exclusively observed during conditions of hypoxia in the EV/2 CA IX expressing cell line. Furthermore, despite high levels of CA IX, virtually no binding of FC11-489A bis occurred after reoxygenation. In 94/1 CA IX knock down cells, no binding of FC11-489A bis was demonstrated irrespective of the oxygen concentration. In conclusion, not only CA IX expression, but also the presence of active CA IX is necessary to enable FC11-489A bis binding, requirements only obtained under hypoxia exposure.

### Example 11

### Effects of a CA IX Inhibitor/Radiation Combination on Cell Survival

Compound S4 (compound 3p from Example 1) was used in this Example to study the effectiveness of CA IX inhibition of the present invention in radiotherapeutic applications.

This compound was tested in a final concentration of 33 μM (in 0.5% DMSO concentration). A stock solution of 330 μM was prepared containing 5% DMSO supplemented with DMEM cell culture growth medium and added on the cells in a 1/10 dilution. For the tests, human colorectal HT-29 adenocarcinoma cells were used harbouring a shRNA against CA IX (94/1) or a scrambled control (EV/2).

### 11.1 Experimental Setup

EV/2 and 94/1 cells were plated at a density of 0.5×10e6 per 6 cm dish (Corning) a day before the start of the experiment and transfer to an anoxic culture chamber (MACS VA500 micro-aerophilic workstation, Don Whitley Scientific, Shipley, UK). The atmosphere in the chamber consisted of 0.0% O₂, 10% H₂, 5% CO₂ and residual N₂. Normoxic dishes were incubated in parallel in air with 5% CO₂. Cells were incubated with S4 1 h after the start of the anoxic/normoxic exposure, during 23 h. Control conditions were obtained by addition of 0.5% DMSO supplemented with medium. After incubation, cells were irradiated on ice (MCN 225 industrial X-ray tube (Philips, Eindhoven, NL) at 225 kV and 10 mA under the respective oxygen concentrations using different doses (Normoxia: 0, 2, 4, 6, and 8 Gy; Anoxia: 0, 4, 8, 12 and 16 Gy). After irradiation, cells were washed, trypsinized and plated for the clonogenic survival assay and incubated under standard culture conditions until colonies were formed (14 days). Colonies were fixed and stained with 4% methylene blue in 70% ethanol. Plating efficiency was determined by counting colonies consisting of >50 cells and correcting for the number of cells seeded.

### 11.2 Results

### 11.2.1 Long-Term Effect of 24 h Anoxia on Survival

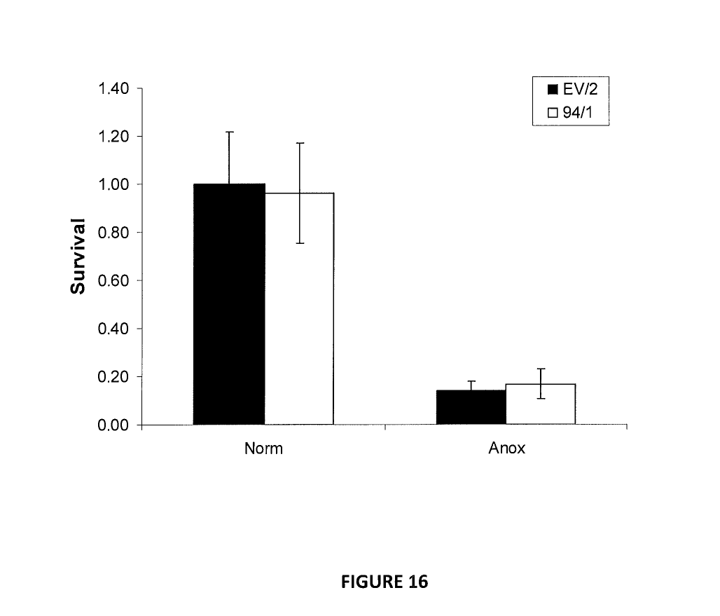

First we investigated if CA IX knock down affected the long-term effect of 24 h anoxia on cell killing, to be able to exclude this effect in the following therapy study. No differences were observed (FIG. 16) between the EV/2 and 94/1 cells regarding their tolerance to low oxygen conditions (P=0.3028).

### 11.2.2 Intrinsic Radiosensitivity

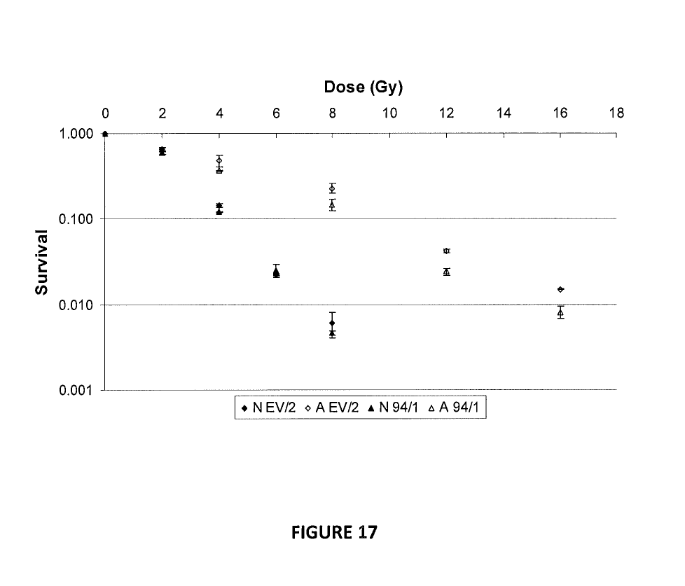

Next, we investigated the effect of CA IX knock down on intrinsic radiosensitivity. FIG. 17 shows intrinsic radiosensitivity of EV/2 CA IX expressing and 94/1 CA IX knock down cells, as assessed using clonogenic survival assay at different irradiation doses. N=normoxia, A=anoxia.

Under normoxic exposure, no differences in survival were found between EV/2 and 94/1 cells regarding their sensitivity to different doses of irradiation. This is in agreement with the equal levels of CA IX mRNA and protein levels under ambient air. However, when cells were exposed to anoxia for 24 h and irradiated under these no oxygen conditions, knock down of CA IX makes cells more sensitive to irradiation (FIG. 17), with a significant difference at 8 Gy (P=0.013), 12 Gy (P<0.0001) and 16 Gy (P=0.0011).

### 11.2.3 S4 Sensitizes CA IX Expressing Cells to Irradiation

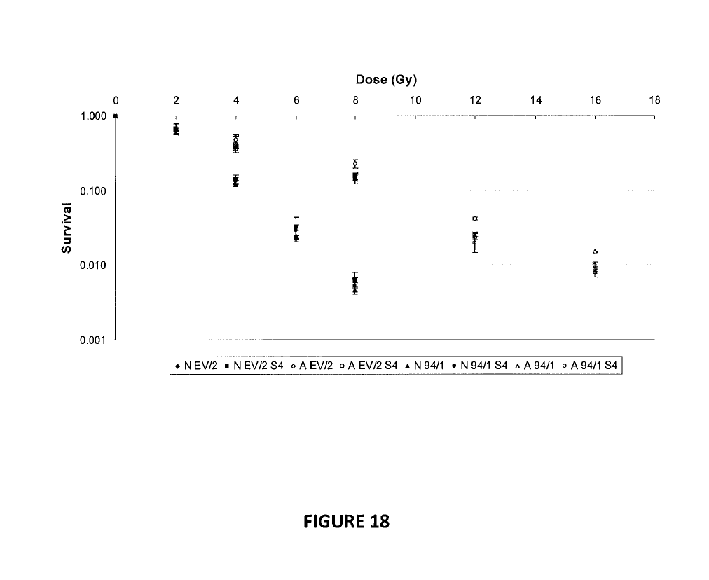

Next, we investigated if the compound S4 is able to sensitize cells to irradiation. Under normoxic exposure, no sensitization to irradiation was demonstrated, neither for the EV/2 and 94/1 cells. When EV/2 CA IX expressing cells were exposed to 24 h anoxia and pretreated with S4, a sensitization to irradiation was observed (FIG. 18) for 8 Gy (P=0.023), 12 Gy (P<0.0001) and 16 Gy (P=0.014). Sensitization of EV/2 cells pretreated with S4 was to a similar extent as seen for the 94/1 CA IX knock down cells without S4 pretreatment, since no significant differences were found between both arms [8 Gy (P=0.6464), 12 Gy (P=0.5067) and 16 Gy (P=0.8691)]. Furthermore, S4 pretreatment of the 94/1 CA IX knock down cells had no effect on the radiosensitivity, demonstrating the CA IX specific inhibition of S4.

### 11.2.4 Conclusion

CA IX inhibition, either using a genetic approach (knock down) or pharmacological (S4) results in a sensitization to irradiation. Genetically or pharmacologically, this sensitization occurs to a similar extent. Pharmacological testing was performed at 33 μM, a concentration selected based on no-toxicity under normoxic conditions based on proliferation/viability assays.

1-29. (canceled)

30. A carbonic anhydrase IX (CA IX) inhibitor which comprises a compound of general formula:
R—NH—CX—NH—(CH₂)ₙ—Ar-Q-SO₂—NH₂
or a pharmaceutically-acceptable salt, derivative or prodrug thereof; wherein
Ar is a substituted or unsubstituted phenylene;
n=0, 1 or 2;
Q is O;
X is O or S; and
R comprises an organic substituent group.

31. A CA IX inhibitor according to claim 30, wherein Ar is a substituted or unsubstituted phenylene or naphthylene.

32. A CA IX inhibitor according to claim 30, wherein the organic substituent group comprises a substituted or unsubstituted cyclic substituent.

33. A CA IX inhibitor according to claim 32, wherein the cyclic substituent comprises an aromatic substituent.

34. A CA IX inhibitor according to claim 33, wherein the aromatic substituent has the formula Ar′—(CH₂)ₚ— in which Ar′ is a substituted or unsubstituted aromatic ring or ring system having up to 3 fused rings and p=0, 1 or 2; or the formula Ar′R′—CH— in which (i) R′ is Me and Ar′ is the same or different and is a substituted or unsubstituted aromatic ring or ring system having up to 3 fused rings, or (ii) R′ and Ar′ are Ph and each Ph is the same or different and is substituted or unsubstituted.

35. A CA IX inhibitor according to claim 33, wherein the aromatic substituent is selected from 4-F—C₆H₄, 4-Cl—C₆H₄, 4-Br—C₆H₄, 4-I—C₆H₄, 2,4,I₂—CH₆H₃, 2,4,6,I₃—C₆H₂, 4-NC—C₆H₄, 4-MeO—C₆H₄, 4-Ph-C₆H₄, 4-PhO—C₆H₄, C₆F₅, 4-PhCH₂—C₆H₄, 4-PhCH₂CH₂C₆H₄, 4-O₂N—C₆H₄, 4-Me₂N—C₆H₄, 2,3,4-F₃C₆H₂, 3,5-Me₂C₆H₃, 4-EtO₂C—C₆H₄, 1-naphthyl, 2-Br-4,6-F₂C₆H₂, 2,4,6-Cl₃C₆H₂, Ph, 3,4-Cl₂C₆H₃, 3-Cl—C₆H₄, 2,4-F₂C₆H₃, 2-Me-4-MeO—C₆H₃, 2-Ph—C₆H₄, 2-PhO—C₆H₄, 3-PhO—C₆H₄, 4-Ac—C₆H₄, 3-Ac—C₆H₄, 4-PhCH₂O—C₆H₄, 2-MeO-5-Me-C₆H₃, 2-EtO—C₆H₄, 4-MeC₆H₄—CH₂, Ph₂CH, 4-iPr—C₆H₄, 2-iPr—C₆H₄, fluoren-9-yl, 3-MeS-C₆H₄, 2-naphthyl, 2-EtOOC—C₆H₄, 3-(2,3-dihydrobenzofuran-5-yl), 3-EtOOC—C₆H₄, 2-NC—C₆H₄, 1-naphthyl-CH₂CH₂, thiophen-2-yl-CH₂CH₂, 3-(2,3-dihydro-benzo[1,4]dioxin-6-yl), furan-2-yl, 1-naphthyl-Me-CH, 3-NO₂—C₆H₄, 2,4(MeO)₂—C₆H₄, 2-Me-4Cl—C₆H₃, Ph-CH₂—CH₂, 4-BuO—C₆H₄, Ph-CH₂, 2-Me-C₆H₄, 2-Cl—C₆H₄, 4-HCOO—C₆H₄, pyridin-2-yl-methyl, pyridin-2-yl-ethyl and pyridine-4-yl-methyl-ethyl.

36. A CA IX inhibitor according to claim 32, wherein the cyclic substituent comprises an alicyclic substituent.

37. A CA IX inhibitor according to claim 36, wherein the alicyclic substituent is 1-adamantyl, N-Boc-piperidin-4-yl or cyclohexyl.

38. A CA IX inhibitor according to claim 30, wherein the organic substituent group comprises a charged moiety.

39. A CA IX inhibitor according to claim 38, wherein the organic substituent is selected from: 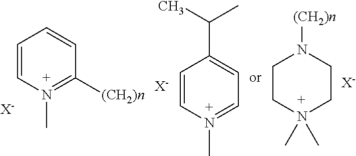
wherein n is 0, 1 or 2; and X is an anion optionally selected from Cl, Br, I and methanesulfonate.

40. A CA IX inhibitor according to claim 32, wherein Ar is para-phenylene.

41. A CA IX inhibitor according to claim 30, wherein X is O.

42. A CA IX inhibitor according to claim 30, wherein n=0.

43. A CA IX inhibitor according to claim 33, which has the following general formula: 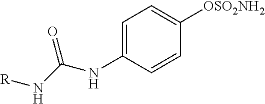
wherein R denotes the aromatic substituent and is selected from 4-F—C₆H₄, 4-Cl—C₆H₄, 4-Br—C₆H₄, 4-I—C₆H₄, 4-NC—C₆H₄, 4-MeO—C₆H₄, 4-Ph-C₆H₄, 4-PhO—C₆H₄, C₆F₅, 4-PhCH₂—C₆H₄, 4-PhCH₂CH₂C₆H₄, 4-O₂N—C₆H₄, 4-Me₂N—C₆H₄, 2,3,4-F₃C₆H₂, 3,5-Me₂C₆H₃, 4-EtO₂C—C₆H₄, 1-naphthyl, 2-Br-4,6-F₂C₆H₂, 2,4,6-Cl₃C₆H₂, Ph, 3,4-Cl₂C₆H₃, 3-Cl—C₆H₄, 2,4-F₂C₆H₃, 2-Me-4-MeO—C₆H₃, 2-Ph-C₆H₄, 2-PhO—C₆H₄, 3-PhO—C₆H₄, 4-Ac—C₆H₄, 3-Ac—C₆H₄, 4-PhCH₂O—C₆H₄, 2-MeO-5-Me-C₆H₃, 2-EtO—C₆H₄, 4-MeC₆H₄—CH₂, Ph₂CH, 4-iPr—C₆H₄, 2-iPr—C₆H₄, fluoren-9-yl, 3-MeS-C₆H₄, 2-naphthyl, 2-EtOOC—C₆H₄, 3-(2,3-dihydrobenzofuran-5-yl), 3-EtOOC—C₆H₄, 2-NC—C₆H₄, 1-naphthyl-CH₂CH₂, thiophen-2-yl-CH₂CH₂, 3-(2,3-dihydro-benzo[1,4]dioxin-6-yl), 1-adamantyl, N-Boc-piperidin-4-yl, pyridin-2-yl-methyl, pyridin-2-yl-ethyl, N-methylpyridinium-2-yl-methyl, N-methylpyridinium-2-yl-ethyl, pyridin-4-yl-methyl-ethyl, N-methyl-pyridinium-4-yl-methyl-ethyl, 4-N-methyl-piperazine-methyl, 4,4-N-dimethyl-piperazinium-ethyl.

44. A CA IX inhibitor according to claim 33, which has the following general formula: 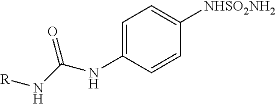
wherein R denotes the aromatic substituent and is selected from
furan-2-yl-CH₂, 3,5-Me₂C₆H₃, 1-naphthyl-CH(CH₃), 3-O₂N—C₆H₄, 2-Me-4-MeO—C₆H₃, 5-Me-2-MeO—C₆H₃, 2-iPr—C₆H₄, 2-Ph-C₆H₄, 2,5-(MeO)₂C₆H₃, cyclohexyl, 2-Me-4-Cl—C₆H₃, 4-PhCH₂CH₂, 4-nBuO—C₆H₄, 4-Cl—C₆H₄, and 4-PhCH₂—.

45. A CA IX inhibitor according to claim 33, which has the following general formula: 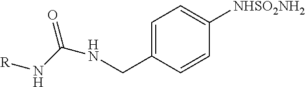
wherein R denotes the aromatic substituent and is selected from Ph, 4-PhCH₂—, 4-MeO—C₆H₄, 4-F—C₆H₄, 4-Me₂N—C₆H₄, 2-MeC₆H₄—, and 2-O—C₆H₄.

46. A CA IX inhibitor according to claim 32, which has the following general formula: 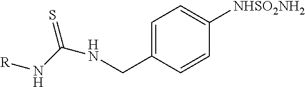
wherein R is selected from CH₂═CH—CH₂—, Ph-, C₆F₅—, CH₃—S—C₆H₄— and 4-(3-Hydroxy-6-oxo-6H-xanthen-9-yl), 3-(HCO₂)C₆H₃—.

47. 4[3,5-dimethylphenyl)ureido]phenyl sulfamate or a pharmaceutically-acceptable salt, derivative or prodrug thereof.

48. 2-(3-hydroxy-6-oxo-6H-xanthen-9-yl)-5-(3-(4-(sulfamoyloxy)phenyl)thioureido) benzoic acid or a pharmaceutically-acceptable salt, derivative or prodrug thereof.

49. A pharmaceutical composition comprising a CA IX inhibitor according to claim 30 and a pharmaceutically-acceptable diluent, excipient or carrier.

50. A pharmaceutical composition comprising
(i) a carbonic anhydrase IX (CA IX) inhibitor, which comprises a compound of general formula:
R—NH—CX—NH—(CH₂)ₙ—Ar-Q-SO₂—NH₂
or a pharmaceutically-acceptable salt, derivative or prodrug thereof; wherein
n=0, 1 or 2;
Q is O or NH;
X is O or S; and
R comprises an organic substituent group; and
(ii) a pharmaceutically-acceptable diluent, excipient or carrier.

51. A product comprising a CA IX inhibitor according to claim 30 and a chemotherapeutic agent as a combined preparation for simultaneous, separate or sequential use in cancer treatment.

52. A product comprising
(i) carbonic anhydrase IX (CA IX) inhibitor, which comprises a compound of general formula:
R—NH—CX—NH—(CH₂)ₙ—Ar-Q-SO₂—NH₂
or a pharmaceutically-acceptable salt, derivative or prodrug thereof; wherein
n=0, 1 or 2;
Q is O or NH;
X is O or S; and
R comprises an organic substituent group; and
(ii) a chemotherapeutic agent, as a combined preparation for simultaneous, separate or sequential use in cancer treatment.

53. A carbonic anhydrase IX (CA IX) inhibitor, which comprises a compound of general formula:
R—NH—CX—NH—(CH₂)ₙ—Ar-Q-SO₂—NH₂
or a pharmaceutically-acceptable salt, derivative or prodrug thereof; wherein
n=0, 1 or 2;
Q is O or NH;
X is O or S; and
R comprises an organic substituent group,
which inhibitor includes a label suitable for use in diagnosis or imaging.

54. A CA IX inhibitor according to claim 53, for use in cancer diagnosis.

55. An imaging composition comprising a CA IX inhibitor according to claim 54 and a suitable diluent, excipient or carrier, wherein the inhibitor includes a label suitable for use in imaging.

56. A method for treating or preventing cancer in a subject in need of such treatment or prevention, which comprises administering to the subject a CA IX inhibitor according to claim 30.

57. A method according to claim 56, which further comprises treating the subject with chemotherapy, radiation therapy or surgery.

58. A method for treating or preventing cancer metastasis in a subject in need of such treatment or prevention, which comprises administering to the subject a carbonic anhydrase IX (CA IX) inhibitor, which comprises a compound of general formula:
R—NH—CX—NH—(CH₂)ₙ—Ar-Q-SO₂—NH₂
or a pharmaceutically-acceptable salt, derivative or prodrug thereof; wherein
n=0, 1 or 2;
Q is O or NH;
X is O or S; and
R comprises an organic substituent group.
